(19) United States
(12) Patent Application Publication
(10) Pub. No.: US 2025/0369801 A1
(43) Pub. Date: Dec. 4, 2025

(54) SYSTEMS AND METHODS FOR FLUORESCENCE-BASED IMAGING

(71) Applicant: The Regents of the University of Michigan, Ann Arbor, MI (US)

(72) Inventors: Joshua Herzog, Ann Arbor, MI (US); Volker Sick, Ann Arbor, MI (US)

(21) Appl. No.: 18/866,184

(22) PCT Filed: Jun. 28, 2023
(86) PCT No.: PCT/US2023/069233

§ 371 Date: Nov. 15, 2024

Related U.S. Application Data
(60) Provisional application No. 63/357,717, filed on Jul. 1, 2022.

(51) Int. Cl. G01J 3/44; G01J 3/02; G01J 3/10; G01J 3/12; G01J 3/28

(52) U.S. Cl. CPC G01J 3/4406; G01J 3/0272; G01J 3/10; G01J 3/12; G01J 3/2803; G01J 3/2823; G01J 2003/1221; G01J 2003/2806

(57) ABSTRACT

A device for two, or more, color fluorescence imaging and methods for fluorescence-based corneal infection imaging. The device includes a frame including an aperture defining an aperture axis, and an imaging lens aligned with the aperture axis. A first image sensor with a first imaging axis aligned with the aperture axis and a second image sensor with a second imaging axis. The device includes a light source configured to emit a light along a source axis, a first beam splitter positioned at an intersection of the aperture axis and the source axis; and a second beam splitter positioned at an intersection of the first imaging axis and the second imaging axis.

### RELATED APPLICATIONS

This application claims priority to and the benefit of U.S. Provisional Patent Application No. 63/357,717 filed Jul. 1, 2022, which is incorporated herein by reference in its entirety for all purposes.

### BACKGROUND

Bacterial and fungal infections of the cornea (microbial keratitis) are clinically challenging conditions that present a significant potential for permanent visual impairment, as well as considerable cost of healthcare resources. Patient outcomes, as well as societal and healthcare costs, depend on the timely diagnosis and treatment of the condition including identification of the infecting microbe and determination of the severity of the infection in addition to the physician's clinical impression. Conventional diagnosis requires the use of laboratory techniques which may be expensive or otherwise inaccessible and specialist training alone is often not sufficient to determine the infecting agent.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a device comprising a frame including an aperture defining an aperture axis, an imaging lens aligned with the aperture axis, a first image sensor with a first imaging axis aligned with the aperture axis, and a second image sensor with a second imaging axis. The device further includes a light source configured to emit a light along a source axis, a first beam splitter positioned at an intersection of the aperture axis and the source axis, and a second beam splitter positioned at an intersection of the first imaging axis and the second imaging axis.

In some embodiments, the device further includes a first filter aligned with the first image sensor, a second filter aligned with the second image sensor, and a third filter aligned with the source axis.

In some embodiments, the device further includes a collimating lens aligned with the source axis.

In some embodiments, the device further includes a cutoff filter positioned between the imaging lens and the first beam splitter.

In some embodiments, the imaging lens is an achromatic lens, a spherical lens, or an aspherical lens.

In some embodiments, the imaging lens is positioned between the first beam splitter and the second beam splitter along the aperture axis.

In some embodiments, the imaging lens has a focal length of 50 mm.

In some embodiments, the imaging lens is transmissive within a range of 400 nm to 700 nm.

In some embodiments, the first filter is a 450 nm long-pass filter.

In some embodiments, the second filter is a 450 nm short-pass filter.

In some embodiments, the first filter is positioned within a threaded aperture of the first image sensor, and the second filter is positioned with a threaded aperture of the second image sensor.

In some embodiments, the second beam splitter has a cutoff wavelength of 458 nm.

In some embodiments, the light source is an ultraviolet light emitting diode.

In some embodiments, the source axis is orthogonal to the aperture axis.

In some embodiments, the source axis is parallel to the second imaging axis.

In some embodiments, the frame includes a first cage, a second cage, a plurality of cage rods extending from the first cage and the second cage.

In some embodiments, the first beam splitter is positioned within the first cage and the second beam splitter is positioned within the second cage.

In some embodiments, the first image sensor and the second image sensor are coupled to the second cage.

In some embodiments, the light source is coupled to the first cage.

In some embodiments, the imaging lens is positioned between the first cage and the second cage.

In some embodiments, the second imaging axis is orthogonal to the first imaging axis.

In some embodiments, a distance between the imaging lens and the first image sensor is 60 mm.

In some embodiments, the device is a hand-held line-of-sight diagnostic tool.

In some embodiments, the device further includes a third image sensor with a third imaging axis, and a third beam splitter positioned at an intersection of the first imaging axis and the third imaging axis.

Another aspect of the present disclosure provides a system for fluorescence-based imaging comprising an aperture configured to be placed in proximity to a biological target. The system further includes a first image sensor configured to capture a first image of the biological target, a second image sensor configured to capture a second image of the biological target, and an imaging lens positioned between the aperture and the first image sensor. The system further includes a light source configured to provide an excitation light to the biological target, a processor configured to analyze the first image and the second image to determine a characteristic of the biological target, and a display configured to display a result of the processor analysis.

In some embodiments, the biological target is an eye, a skin sample, or a cell sample.

In some embodiments, the characteristic of the biological target determined is an optical redox ratio or luminescence intensity ratio.

In some embodiments, the biological target is an eye and the characteristic includes the presence of a microbial infection, or the presence and severity of a cataract, or age-related lens chemistry, or UV-related lens chemistry.

In some embodiments, the system further includes an adjustment assembly to adjust the position of the aperture relative to the eye.

In some embodiments, the system further includes a first beam splitter and a second beam splitter.

In some embodiments, the biological target is an eye and the system further includes an adjustable eyepiece coupled to the aperture.

In some embodiments, the system further includes a first filter aligned with the first image sensor, a second filter aligned with the second image sensor, and a third filter aligned with the source axis.

In some embodiments, the system further includes a sensor to monitor the output of the light source.

In some embodiments, the system further includes a plurality of calibration targets.

In some embodiments, the first image sensor is configured to capture a plurality of images to form a first composite image.

In some embodiments, the second image sensor is configured to capture a plurality of images to form a second composite image.

In some embodiments, the system further includes a third image sensor configured to capture a third image of the biological target, and wherein the processor is configured to analyze the first image, the second image, and the third image to determine the characteristic of the biological target.

Another aspect of the present disclosure provides a method of detecting corneal infection comprising providing a device with an aperture configured to be placed in proximity to an eye, a light source, a first image sensor, and a second image sensor, and illuminate the eye with an excitation light from the light source. The method further includes collecting a first image of the eye with the first image sensor, collecting a second image of the eye with the second image sensor, and analyzing the first image and the second image to determine whether the eye has an infection.

In some embodiments, analyzing the first image and the second image includes identifying structures of the eye and identifying boundaries between ulcerated regions of the eye and healthy regions of the eye.

In some embodiments analyzing further includes classifying ulcerated regions of the eye as fungal, bacterial, or uninfected.

In some embodiments, analyzing further includes classifying a species of fungus or bacteria present in the eye.

In some embodiments, analyzing further includes determining size, shape, location, and severity of the ulcerated regions of the eye.

In some embodiments, analyzing further includes classifying the ulcerated regions according to size, shape, location, or pathogen.

In some embodiments, the method further includes determining a diagnosis based on the analysis of the first image and the second image.

In some embodiments, the method further includes reporting the diagnosis to a user.

In some embodiments, the method further includes estimating from the healthy regions of the eye: a pupil radius, a pupil eccentricity, a pupil irregularity, a lens fluorescence quantum yield, a lens luminescence intensity ratio, an iris inner radius, an iris outer radius, an iris eccentricity, or an iris border irregularity.

In some embodiments, a plurality of exposures is collected from each camera and are used to form a composite exposure.

In some embodiments, for each of the plurality of exposures, the aperture is at a different position with respect to the eye.

In some embodiments, collecting the first image and the second image is with the device in a first position relative to the eye, and wherein the method further includes collecting a third image of the eye with the first image sensor and a fourth image of the eye with the second image sensor with the device in a second position relative to the eye.

In some embodiments, the first image and the second image are captured with a spatial resolution of 5 mm⁻¹.

In some embodiments, analyzing the first image and the second image includes calculating a ratio image such that the ratio measured at each pixel is equal to the spectral luminescence intensity ratio normalized to a reference condition.

In some embodiments, the excitation light includes 370 nm excitation.

In some embodiments, the method further includes collecting a third image of the eye with a third image sensor of the device, and analyzing the first image, the second image, and the third image to determine whether the eye has an infection.

Other aspects of the disclosure will become apparent by consideration of the detailed description and accompanying drawings.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIG. 1A is an illustration of a diagnostic device with an excitation source (e.g., a LED) and two image sensors (e.g., cameras).

FIG. 1B is an illustration of a diagnostic device with an excitation source (e.g., a LED) and three image sensors (e.g., cameras).

FIG. 2 is a perspective view of a diagnostic device according to one embodiment of the invention.

FIG. 3 is a section view of the diagnostic device of FIG. 2.

FIG. 4 is a graph of calculated exposure limits for 370 nm excitation based on ICNIRP guidelines as a function of exposure duration for a single exposure, with an estimated exposure for a 1.5 W LED superimposed.

FIG. 5 is a graph of maximum allowed duty cycle as a function of pulse count for several exposures at 37.5 mW/cm² incident optical power.

FIG. 6 is an optical layout for the LED collimation optics using a lens of focal length f. The black arrows of FIG. 6 indicate the source and target (e.g., cornea) locations.

FIG. 7 includes sample fluence profiles calculated using EQN. 20 for S′_{q}=300 mm, d=25 mm, and r=1.5 mm, and variable S′ₚ using two different lens focal lengths. Left: f=25 mm. Right: f=20 mm.

FIG. 8 is a graph of calculated fluence profile for several different source lengths and f=25 mm, S′_{q}=300 mm, d=25 mm, and S′ₚ=26 mm.

FIG. 9 is calculated PSFs for the achromatic (e.g., Edmund Optics #65-976) and aspherical (Edmund Optics #33-945) lenses at several wavelengths. For the calculation, the image plane and object plane distances are fixed at 60 mm and 300 mm from the principal plane, respectively.

FIG. 10 is calculated intensity profiles at cornea plane for the assumed source geometry with different collimating lens focal lengths.

FIG. 11 is calculated image and ratio signal-to-noise ratio on a per-pixel basis as a function of effective microbe layer thickness at a fixed exposure duration. The solid curves indicate the SNR in the absence of fluorescence from the eye. The dashed curves include additional shot-noise resulting from intrinsic eye fluorescence, but otherwise assume the background subtraction is perfect.

FIG. 12 is calculated PSFs showing lateral chromatic aberrations for both imaging bands. The plots labeled ‘lateral’ indicate the PSF of a point at a height hₒ from the optical axis averaged over wavelength, while the ‘chromatic’ plot indicates the PSF of a source with wavelength λ averaged over image heights.

FIG. 13 is calculated PSFs showing axial chromatic aberrations for both imaging bands. The plots labelled ‘axial’ indicated the PSF on a point at a distance ΔSₒ from the object plane along the optical axis averaged over wavelength, while the ‘chromatic’ plot indicates the PSF of a source with wavelength λ averaged over object distances.

FIG. 14 is average PSF for polychromatic point sources off the optical axis in the object plane FIG. 13a, and along the optical axis near the object plane FIG. 13b

FIG. 15 is calculated irradiance at the cornea plane from a 2W LED. The LED is placed 20 mm behind the lens front surface and the cornea plane is 290 mm behind the lens back surface.

FIG. 16 is SLIR of B. subtilis and green bread mold fungus normalized to that of E. coli as a function of cutoff wavelength at 340 nm and 370 nm excitation.

FIG. 17 is graph of calculated error metric E as a function of cutoff wavelength for 340, 365, and 370 nm excitation wavelengths.

FIG. 18 is a graph of transmission/intensity as a function of wavelength for a device with two imaging bands, two imaging filters, and an excitation bandpass filter. Transmission spectra calculated for the red and blue imaging bands and the LED band based on manufacturer-provided data. The LED emission profile and measured B. cereus and S. marcescens fluorescence spectra are superimposed.

FIG. 19A is a graph of sample luminescence intensity ratio at different excitation wavelengths and relative mole fractions calculated from a combined NADH/FAD model.

FIG. 19B is a graph of sample ratio sensitivity to NADH/FAD mole fraction ratio at different excitation wavelengths and relative mole fractions calculated from a combined NADH/FAD model.

FIG. 20A and FIG. 20B are graphs of calculated measurement limits and imaging performance (S_{χ}/χ for htn=10¹⁴ s/cm²) for mixtures of NADH and FAD.

FIG. 21 is a graph of calculated COV for fluorescence signal and FIR for a B. subtilis smear as a function of thickness h and integration duration t using the diagnostic strategy according to one embodiment.

FIG. 22A and FIG. 22B illustrate sample imaging results for 8 species.

FIG. 23 is a graph of joint PDF and FIR and corrected blue-band emission intensity rate for each sample.

FIG. 24 is a perspective view of a positioning assembly for an imaging device.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “processor” (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment, and can be configured to communicate via wired or wireless communications with other processors, where such one or more processor can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non-transitory computer readable medium such as another memory or a disc. In some embodiments, the memory includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

Unless otherwise defined, all technical terms used herein have the same meaning as commonly understood by one of ordinary skill in the art to which this disclosure belongs.

Many conventional imaging modalities exist that have been used generally for analysis of tissue and cell samples, and specifically for detection or distinguishing bacteria and fungi in vitro. Methods rely on multi- or hyper-spectral imaging (MSI or HSI) which, while having the potential for extremely high accuracy, is expensive and impractical for some purposes. HSI can be applied using conventional point measurement spectroscopy techniques including Fourier transform infrared (FTIR) absorption spectroscopy, near-IR (NIR) absorption spectroscopy, and spontaneous Raman spectroscopy, each of which has been used successfully for detection and identification of bacterial and fungal samples. While potentially more accurate, HSI techniques add significantly to the cost and complexity of the device. Further, due to the nature of HSI, measurements would necessarily increase the required radiation exposure, increasing the risk to the patient.

Absorption techniques, e.g., NIR or mid-infrared (MIR) absorption, could be performed using a two excitation-wavelength strategy without the need for HSI, using a diffuse backlight imaging (DBI) approach. However, this would require the use of IR-sensitive cameras and optics, increasing the cost and complexity of the device. Further, in vivo NIR radiation exposure of, for example, the eye, tends to be more damaging; and for an equivalent excitation and spatial resolution, the signal-to-noise ratio (SNR) of an DBI absorption diagnostic is not expected to be improved over that of a fluorescence-imaging measurement. Finally, there are additional technical challenges associated with DBI, namely the generation of the diffuse backlight that could be accomplished using the diffuse reflection from the eye (typical diffuse reflectance is on the order of 5-10% at red to near-IR wavelengths), but this reflection will have some dependence on the structure of the individual eye and will not be uniform. Additional challenges result from specular reflection, changes in reflectivity due to corneal ulcers, and the curvature of the cornea. A similar measurement utilizing the wavelength-dependence of elastic scattering, particularly at infrared or near-UV wavelengths where significant absorption is expected, may also be feasible; however, it is not clear how sensitive elastic scattering spectra are to bacterial or fungal species. There is additionally an incidence angle dependence to scattering spectra, leading to further experimental complexities or uncertainties.

Spontaneous Raman scattering is likely not feasible for bacterial and fungal imaging in patients due to the prohibitively low scattering cross-sections, which are typically several orders of magnitude weaker than molecular absorption cross-sections. Raman scattering measurements would thus require either long exposure times or high-intensity light sources. Similarly, nonlinear spectroscopy techniques, including two and three-photon absorption (2PA and 3PA) fluorescence imaging and coherent Raman scattering (CRS) are not believed to be feasible because they require high instantaneous-power excitation which could pose a significant risk of eye damage.

Fluorescence imaging, on the other hand, is relatively inexpensive, fast, and distinct. Fluorescence of chemicals that exist within bacterial and fungal cells can be excited at near-UV wavelengths, typically emit with similar spectra at blue to green wavelengths, but are sufficiently distinct that genus classification can be made based on fluorescence emission. Since excitation can be performed at near UV wavelengths with visible emission bands, relatively low-cost optical equipment can be leveraged. Further, since the emission wavelength is spectrally-shifted with respect to the excitation source, background scattering and reflections can be easily avoided.

As disclosed herein are new, non-intrusive diagnostic technologies for conditions of the eye, including microbial keratitis, that can be performed without specialist training, have potential to reduce access barriers, improve patient outcomes, and lower healthcare costs. For example, a fluorescence-based imaging tool is described herein. A low-cost fluorescence-imaging diagnostic tool that is capable of detecting and distinguishing bacterial and fungal infections of the cornea and conjunctiva is detailed herein. The diagnostic can be entirely self-contained and will not require specialist training to operate. The device uses ultraviolet-visible (UV-vis) fluorescence spectroscopy; a UV light source is used to excite microbial cells, and a camera is used to image the resulting fluorescence, providing a map of the patient's eye showing, e.g., relative concentrations of bacterial or fungal cells in suspected cases of microbial keratitis.

The solution described herein utilizes the fluorescence of naturally-occurring tracer molecules (e.g., NAD(P)H, FAD, ergosterol, tryptophan, collagens, melanins) found in all cells. Fluorescence-based diagnostics are an attractive tool for medicine since they are typically non-invasive, portable, and can provide unique visual information that is otherwise inaccessible to the physician but are only recently being applied to medical diagnosis. Fluorescence refers to the fast emission of light following absorption of light at a shorter wavelength. The wavelengths at which a material absorbs, and those at which it emits, depend on the material. These wavelengths can be used for “molecular fingerprinting”, potentially allowing for trace detection and identification of, for example, microbes in cases of microbial keratitis.

Several molecules exist that can be excited and detected in bacteria and fungi using UV excitation. In particular, the reduced form of nicotinamide adenine dinucleotide (NAD; NADH in reduced form) is a UV-active coenzyme that exists in all cells including bacteria and has known fluorescence properties. Fluorescence emission of E. coli in water has directly been attributed to NADH. Similarly, ergosterol, a primary component in fungal cell membranes and target of many clinically available antifungal drugs, is another UV-active molecule that has been studied spectroscopically in the past. Tryptophan also has similar emission and absorption features as ergosterol and has been detected in E. coli and other bacterial samples. Flavoproteins, and flavin adenine dinucleotide (FAD) in particular, absorb near-UV and blue light and emit at green wavelengths and are found in a wide variety of cells. Finally, in other tissues and microbial species, various melanins and collagens fluoresce when excited at near-UV wavelengths. The relative emission intensity, or ratio of emission intensities (the luminescence intensity ratio), of any pair of these fluorescent molecules is a characteristic of the biological sample.

Flavin adenine dinucleotide (FAD) and reduced nicotinamide adenine dinucleotide (NADH) play a significant role in cell metabolism and can be probed optically; for this reason they have been targeted across a variety of cell and tissue types to measure metabolic function and detect various tissue pathologies by taking advantage of the fluorescence of NADH and FAD. The NADH to FAD fluorescence ratio in particular, often referred to as the redox ratio, is used as a general measure of metabolic activity in a variety of applications including cancer tissue analysis, monitoring wound healing, and in a variety of other cell metabolism studies.

Devices disclosed herein for rapid screening or evaluation of samples provide a robust and cost-effective means for, e.g., pre-clinical drug screening, evaluation of wound or infection healing, detection of surface contamination, diagnosis of cancer, and evaluation of mitochondrial dysfunction.

Similarly, fluorescence spectroscopy using NADH and FAD as probes can be used for the detection and identification of microbe species. Fluorescence intensity and spectra (likely due at least in part to NADH) have been shown to be feasible for evaluating bactericide efficacy, and in some embodiments, the redox ratio is used for identifying bacterial species in urinary tract infections.

Confocal and multiphoton microscopy techniques are often used for fluorescence studies of biological samples in clinical settings because they can be used to construct a three-dimensional fluorescence map with high spatial resolution. Confocal and multiphoton microscopy can achieve high spatial-resolution by using point illumination and exploiting optical nonlinearity, respectively, and multiphoton microscopy in particular can reach penetration depths on the order of 1 mm in biological tissues. However, both techniques can be slow and have limited fields of view resulting from the need to scan the illumination source. There is significant utility in the rapid evaluation of redox ratio or similar measures for the identification of biological cultures and in characterizing tissue samples. However, most quantitative methods reported to date require relatively specialized and expensive equipment, and specialized training. While accurate, these methods can be expensive and inaccessible in many settings, making them largely unsuitable for rapid, low-cost analysis and medical imaging outside of well-equipped laboratories.

The diagnostic strategy disclosed herein is a single excitation-wavelength, dual-imaging band fluorescence-based diagnostic. Briefly, a single narrow-band UV light source is used to excite microbes in the patient's eye or other sample at a wavelength of approximately 370 nm, for example. In some embodiments, the UV light source has a wavelength of approximately 340 nm. In some embodiments, the UV light source has a wavelength of approximately 315 nm. In some embodiments, the UV light source has a wavelength within a range of approximately 280 nm to approximately 300 nm. This excites blue NADH and green FAD fluorescence in many bacteria and fungi and in the eye. A camera with an appropriate set of short-pass long-pass filters is then used to image the fluorescence in two emission bands corresponding to NADH and FAD. The measured fluorescence images are normalized using a flatfield correction procedure such that the resulting image depends only on the relative concentrations and states of the excited species. Concentration maps of bacteria and fungi are inferred from the images and these quantities are used to determine the severity and extent (or absence) of a microbial infection in suspected cases of microbial keratitis. The ratio of the concentration images is a function of the infecting agent only, and can be used to distinguish between sources of infection. Other physiological characteristics or abnormalities (e.g., cataracts, physiochemical changes in the lens, and conjunctival autofluorescence) may be detected in a similar fashion.

As such, the device discloses herein is relatively low-cost and easy to manufacture. The device includes a UV light source, two scientific cameras or image sensors, an imaging lens, thin-film interference filters, and beam splitters. Since the fluorescence emission is visible, conventional low-cost CMOS image sensors are sufficient for this application.

Excitation of microbes in vivo requires exposure of patients to UV radiation, which is known to cause adverse biological effects at high exposures. However, the proposed diagnostic is relatively low risk. The primary mechanism of damage from near-UV exposure in the human eye is thermal rather than photo-chemical, and as a result incidental exposure limits for UV radiation are typically larger than visible or infrared (IR). The diagnostic imaging strategy will be designed to work within the International Commission on Non-Ionizing Radiation Protection (ICNIRP) recommended incidental exposure limits and limit UV exposure.

Two bacteria samples (Escherichia coli pYAC4, and Bacillus subtilis PY79) were characterized as somewhat diverse examples of bacteria (E. coli is a Gram-negative rod, while B. subtilis is a Gram-positive rod). Both pYAC4 and PY79 are non-pathogenic, although cases have been reported in which patients have tested positive for both B. subtilis and E. coli keratitis. In addition, a representative fungal sample, cultured from green bread mold fungus, was tested for comparison. Characterization of samples included relative fluorescence emission spectrum using two excitation sources (340 nm and 370 nm), and absolute fluorescence emission intensities (using the 340 nm source) at a fixed excitation fluence.

The proposed diagnostic technique uses the luminescence emission following excitation by an external ultraviolet source. Thus, the fluorescence emission spectra of the different microbes should be distinguishable to determine the infecting agent. Measurements of the relative emission spectra of three microbial samples were made to identify spectral features that could be used to distinguish between samples.

Based on the observed emission spectra dependence on species, a ratiometric technique is proposed for the diagnostic. Briefly, two images are taken of the fluorescence emission using two different transmission bands (labelled “red” and “blue”, corresponding to the long wavelength and short wavelength regions, respectively). A background subtraction is performed for each band, and a flatfield correction procedure is used to account for variation in lens collection efficiency and excitation fluence. Finally, a ratio image is calculated from the corrected images. In this way, the ratio measured at each pixel is equal to the spectral luminescence intensity ratio (SLIR) normalized to a reference condition. The SLIR is a property of the emission spectrum; it is dependent only on properties of the microbial sample and is independent of the experimental setup. Mathematically, the SLIR is calculated from the image data as EQN. 1.

$${R}_{ij} = \frac{{S}_{2 , ij} - {B}_{2 , ij}}{{S}_{1 , ij} - {B}_{1 , ij}} \frac{{S}_{1 , ij}^{( ref )} - {B}_{1 , ij}^{( ref )}}{{S}_{2 , ij}^{( ref )} - {B}_{2 , ij}^{( ref )}} = \frac{{S}_{2 , ij} - {B}_{2 , ij}}{{S}_{1 , ij} - {B}_{1 , ij}} \frac{1}{{R}_{ij}^{( ref )}} \qquad EQN . 1$$

where S_{k,ij} is the measured signal (in photons) and B_{k,ij} is the measured background signal in band k at pixel location (i, j). The band labels 2 and 1 correspond to the “red” and “blue” bands, respectively, and the subscript label (ref) refers to the reference of flatfield correction condition. On the right hand side of EQN. 1, the flatfield correction is rewritten such that the correction is applied to the ratio image rather than the intensity images.

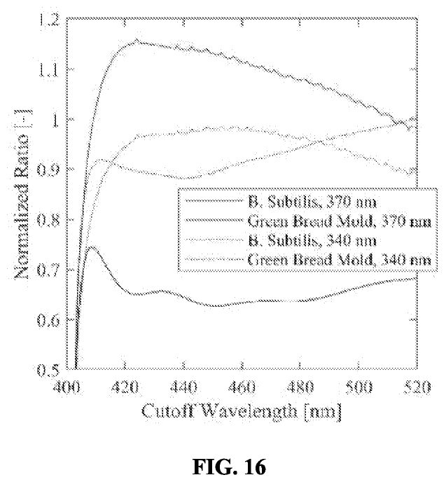

The SLIR can be estimated from an emission spectrum according to EQN. 2.

$$R = \frac{{\int }_{{λ}_{c}}^{∞} d λ I ( λ )}{{\int }_{0}^{{λ}_{c}} d λ I ( λ )} \qquad EQN . 2$$

Where λ represents the emission wavelength, I is the emission spectrum of the sample, and λ_{c} is the cutoff wavelength separating the red and blue bands. The SLIRs for B. subtilis and the bread mold fungus normalized by the SLIRs for E. coli, are plotted as function of cutoff wavelength c in FIG. 16. The ratios presented here consider the luminescence between 400 and 535 nm. From FIG. 16, the largest variation between samples occurs with 370 nm excitation and is not strongly dependent on cutoff wavelengths between 420 nm and 480 nm. There is a significant difference (e.g., >10%) in the SLIR at 370 nm between all of the samples.

With reference to optimization of collection bands, in order to distinguish between microbial samples the SLIR should vary from sample to sample. The collection bands should also be wide enough to provide enough signal and ensure the precision to which the ratio is measured is sufficient for detection. The ratio precision is estimated from first-order uncertainty propagation as EQN. 3.

$$\frac{{s}_{R}^{2}}{{R}^{2}} = \frac{{s}_{{S}_{1}}^{2}}{{S}_{1}^{2}} + \frac{{s}_{{S}_{2}}^{2}}{{S}_{2}^{2}} = \frac{1}{{S}_{1}} + \frac{1}{{S}_{2}} \qquad EQN . 3$$

Where the symbol sₓ represents the precision index (e.g., single-shot standard deviation) in the variable x. ON the right hand side, it assumed the diagnostic is shot-noise limited (sₛ_{ᵢ}t=\sqrt{Sᵢ}). Rewriting using the definition of the ratio provided in EQN. 4.

$$\frac{{s}_{R}}{R} = \frac{\sqrt{1 + R}}{{SNR}_{2}} \qquad EQN . 4$$

Where SNR₂=S₂/sₛ_{₂} is the signal-to-noise (SNR) ratio of the red band. Assuming the entire emission spectrum is collected between the two cameras (in this example, from 400 to 535 nm), with fraction f being collected in the red band, then the ratio and precision can be rewritten further as EQN. 5 and EQN. 6.

$$R = \frac{f}{1 - f} \qquad EQN . 5$$

$$\frac{{s}_{R}}{R} = {{c}^{- 1} ( f - {f}^{2} )}^{- \frac{1}{2}} \qquad EQN . 6$$

Where c=SNR=\sqrt{S} is the SNR (or square-root of the total number of collected photons) if the entire emission band were captured on a single detector.

To optimize diagnostic performance, relative error E=cs_{R}/ΔR where ΔR is the expected change in the ratio should be minimized. As such, the cutoff wavelength/c that minimizes E is determined. Because multiple species are considered in some embodiments, ΔR is calculated as the root-mean-square of the SLIR difference between every pair of species and s_{R} is averaged over each species. The diagnostic performance based on the observed E. coli, B. subtilis, and mold fluorescence spectra (assuming the total number of collected photons is unchanged) is advantageous with approximately 370 nm excitation, and diagnostic precision peaks at a cutoff wavelength of approximately 455 nm.

In addition to spectroscopy of the microbial samples, several additional factors are considered to design an effective diagnostic in addition to the basic optical requirements. For example, diagnostic performance is directly related to temporal and spatial resolution which results in a performance trade-off. Error due to motion must also be considered as the human eye is a dynamic system that moves even while remaining fixated on a target. If the device is portable and handheld, there are restrictions on lens focal length, depth of field, and sensor positioning. Similarly, if the lenses are fixed in place, a method to focus the diagnostic on the patient's eye is presented herein. Finally, any active-imaging tool that requires UV-excitation should consider intrinsic eye fluorescence and the impact of the UV exposure on the patient's eye.

The solutions presented herein include a tool that can rapidly diagnose conditions of the eye including microbial keratitis and can be used by non-specialists with minimal training. The tool described herein enables providers or technicians that are not eye or cornea specialists to rapidly diagnose the presence of a bacterial or fungal infection in the cornea (or lack thereof). As an imaging diagnostic, this provides the capability to store and transmit image series showing relative microbial concentration, which can be used to identify the shape or extent and severity of the infection in addition to the infecting microbe. The solution presented herein is for widely accessible screening for corneal infections and ulcers; can diagnose and monitor corneal infections by non-specialists and mobile technicians; identify infecting microbes in an accurate, rapid, and non-intrusive manner; and records quantitative data on severity and extent of infection that can be transmitted to specialists for remote analysis. The solution presented herein is portable, low-cost, includes convenient and automated operation, optical and non-contact (e.g., non-invasive and rapid), spatially resolved field measurement (imaging), and automated analysis.

In one embodiment, the tool disclosed herein uses two nominally identical CCD or CMOS cameras, a low-cost UV LED, a series of beam splitters and imaging optics to implement a ratiometric imaging technique according to the results presented herein. Ratiometric imaging is a multispectral imaging technique in which two wavelength bands are captured simultaneously and the SLIR is calculated. This provides a relatively low-cost and low-complexity spectroscopic imaging technique that has the capability of detecting and distinguishing pathogens while simultaneously allowing for straightforward operation and data analysis.

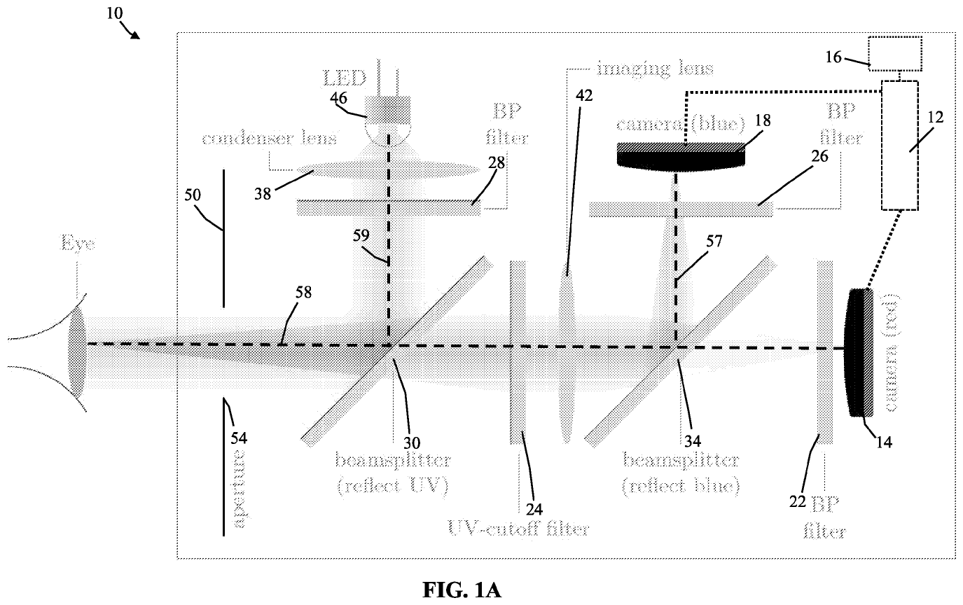

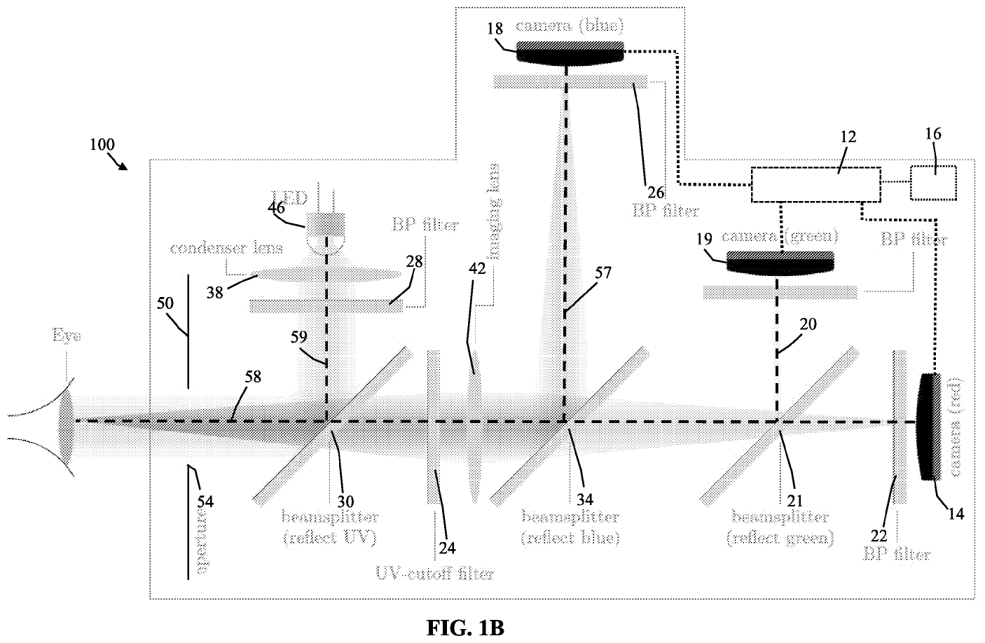

With reference to FIG. 1A, an optical layout of the device 10 is shown with the relative locations of the two image sensors or cameras 14, 18, the optical filters 22, 24, 26, 28, the beam splitters 30, 34, the lens 38 used for LED collimation, and the lens 42 used for image formation. The image and object plane distances (from the imaging lens plane) are given by Sᵢ and Sₒ, respectively, and these determine the magnification of the imaging system. A beam path is illustrated of the excitation source 46 (e.g., the light source, the LED) and a ray path is illustrated of the emission from a point on the object plane. In the illustrated embodiment, the device 10 includes frame 50 with an aperture 54 that defines an aperture axis 58. In the illustrated embodiment the aperture axis 58 is an optical axis.

To keep cost relatively low and to minimize the footprint of the device, a fixed singlet lens 42 is utilized. Focusing is achieved by translating the entire device 10 along an adjustment assembly (e.g., a track), and the focal point is determined by imaging fluorescence of the cornea after a field stop is applied to the LED to provide a sharp cutoff (eye fluorescence and focus will be discussed in more depth herein). In some embodiments, exposure, image capture, and image analysis will be performed by an on-board computer or processor 12. In some embodiments, a display 16 is included to display a result (e.g., infection detected) of the processor 12 analysis. In some embodiments, the display 16 is integrated into the handheld device 10. In some embodiments, the display 16 is a LCD screen or a touch screen (e.g., a touch screen of a portable cell phone).

As such, the device 10 provides a two-camera, line-of-sight near-UV ratiometric fluorescence imaging tool. Unlike typical ratiometric imaging setups, the two cameras 14, 18 share an achromatic lens 42 which ensures the image plane distance and camera magnification is fixed between the two cameras. The UV excitation source 46 is placed in front of the imaging lens and provides illumination along the line-of-sight direction. Two dichroic beam splitters 30, 34 are used in the design; the first is used to direct the UV source towards the object plane while transmitting fluorescence light, and the second is used to separate the fluorescence image between the two image sensors with a cutoff wavelength. The fluorescence intensity ratio (FIR) is calculated as the ratio of the two fluorescence images.

With reference to FIG. 1B, in one embodiment, a device 100 includes the same or similar components as the device 10 and similar reference numerals are utilized to reference the same. The device 100 further includes but also includes a third image sensor 19 with a third imaging axis 20, and a third beam splitter 21 positioned at an intersection of the first imaging axis 58 and the third imaging axis 20. The third image sensor 19 is configured to capture a third image of the biological target, and the processor 12 is configured to analyze the first image, the second image, and the third image to determine the characteristic of the biological target.

A few design parameters that influence performance include spatial extent, depth of field, lens f-number, focal length (for a fixed aperture size), spatial resolution (or object-plane pixel size), exposure duration, and LED irradiance. The spatial extent and depth of field are determined by geometrical optics and are chosen to allow the entire cornea to be imaged. Because the device 10 uses CW-excitation and fluorescence, the measurement SNR is directly related to the remaining parameters. For a uniform pathogen surface concentration in the shot-noise limit, the SNR scales as EQN. 7.

$$SNR ∝ \frac{\sqrt{{t}_{exp}} {l}_{p} I}{{f}_{\#}} \qquad EQN . 7$$

Where tₑₓₚ is the exposure duration, lₚ is the effective object-plane pixel size, f_{#} is the lens f-number, and I is the excitation irradiance. Since this is a line-of-sight imaging technique, the out-of-plane resolution does not strongly impact the diagnostic and can be ignored.

Assuming fluorescence is dominated by intrinsic cornea and lens fluorescence, the signal-to-background ratio (SBR) to first order is dependent only on the pathogen concentration and type as both components scale identically with exposure, irradiance, and pixel size. Performance is thus maximized when the SNR is maximized. Mathematically, the exposure, irradiance, and pixel size are maximized while the f-number should be minimized subject to a set of reasonable constraints that will be discussed further herein.

The extent of the imaging region is largely determined by the size of the cornea. The average horizontal diameter of the cornea is approximately 12 mm although measurements of approximately 13 mm have been reported. Thus, the minimum length of the imaging region is at least approximately 15 mm to account for the size of the cornea and possible alignment error. The cornea additionally has an average radius of curvature of 7.8 mm; the edge of the cornea (at a radius of 5.75 mm from the vertex) sits approximately 2.5 mm behind the vertex point, requiring a depth of field of at least 2.5 mm. The depth of field distance of at least 2.5 mm is large compared to the cornea thickness of approximately 0.5 mm.

Equally important is variation in the depth of the eye surface in relation to the deepest depression of the nasal bones or sellion (hereafter referred to as “cornea depth”) from patient to patient. This distance is related to the vertex distance, which is the distance between the anterior surface of the cornea and the back surface of a corrective lens. In some embodiments, the device 10 focus is fixed, the device 10 translates physically to account for this variation. Here we assume that any variation in cornea depth between individuals is equal to the variation in vertex distance. Limited information is available on this distribution, although it has been measured and reported that the distance varies from approximately 8 to 20 mm with a mean of 13 mm and standard deviation of around 2 mm. Assuming the system makes use of an eyepiece that is placed directly against the face, the combined depth of field and translation distance of the device must be at least approximately 15 mm.

Finally, most patients are not able to keep their heads perfectly still, particularly while in pain. Some measurements of typical unconstrained head motion include a typical mean absolute deviation measured over a 100 second period of approximately 1.5 mm, with maximum deviations around 2 mm. Deviations at the 1 second time scale are much smaller and are not expected to influence image resolution or cause motion blur during exposure. Since the proposed procedure is expected to take less than 100 s including multiple image acquisitions and focusing, the 2 mm maximum deviation is believed to be a reasonably strict upper bound on axial head motion and positioning error.

In summary, the depth of field is at least approximately 5 mm to account for head motion, cornea curvature, and small positioning errors. The minimum imaging region size is at least 18 mm square to account for the size of the cornea and some slight translation error due to head motion. Finally, the system or device translate along the optical axis approximately 20 mm (10 mm in either direction) to allow for focusing.

There is a tradeoff between spatial resolution, temporal resolution, and performance. Generally, it is desired to maximize performance (imaging SNR) while maintaining a target spatial resolution. For a fixed time-invariant system such as the eye, temporal resolution is not the most important and instead the maximum feasible exposure duration is chosen to maximize imaging performance.

The state of the art is not settled on what spatial resolution is required for diagnosing and monitoring infections of the eye. A recent study showed that slit lamp caliper measurements of epithelial defect and stromal infiltrate size (both height and width) made by different cornea specialists varied by at least 0.5 mm in roughly one third of cases. This provides an estimate of the spatial resolution with which measurements are made currently. A target spatial resolution is chosen in the design disclosed herein as 5 lp/mm (such that 0.2 mm features are resolvable) to provide a significant improvement over the current limit. Assuming a perfectly sampled imaging system, the target object-plane pixel size is taken to be 0.1 mm in order to satisfy the Nyquist-Shannon sampling criterion.

While temporal resolution does not directly impact the result since the eye is approximately a steady state system, it can indirectly impact spatial resolution through eye fixation error. “Fixation” refers to maintaining the orientation of the eye on a visual target for an extended period. This process requires active ocular stabilization and is therefore subject to error, primarily in the form of random fluctuations. This fixation error has been characterized in several studies. Orientation error (over a 15 s period) is similar in the vertical and horizontal directions with typical values of 0.1° with variation of around 50% between subjects. This is the result of several different motions. At the largest scale, slow drifts occur on the order of a second taking the eye away from the fixation target, followed by a rapid restoration within 25 ms (a “saccade”). This occurs in addition to a small, random fluctuating component (“tremor”).

Assuming a maximum orientation error of 0.2°, corresponding to a translation error of 50 μm, this fixation error is well within the target spatial resolution and measurement times on the order of several seconds for healthy individuals appear to be acceptable. However, this result does provide a lower bound on spatial resolution of 50 μm. To improve spatial resolution beyond this value, it is necessary to reduce the exposure duration accordingly. Since this error is largely a result of the drift motion, which has approximately constant velocity over a 0.5 second period, the required exposure Δt for a spatial resolution Δx can be estimated as EQN. 8 for Δt less than or approximately equal to 1 second.

$$\frac{Δ x}{Δ t} \geq 100 μm / s \qquad EQN . 8$$

Regarding intrinsic eye fluorescence, the cornea and lens tissue absorb near-UV light and emit with a peak near 450 nm similar to the observed microbe emission spectra. It has been suggested that fluorescence excited at 340 nm excitation in cornea tissue is primarily due to the NADH and NADPH molecules, which are two of the same molecules believed to be responsible for the observed bacteria and fungi fluorescence. There appears to be no direct measurements of pyridine nucleotide concentrations in human corneas. However, human lens tissue contains approximately 50 nmol/g NADH and 10 nmol/g NADPH. In the absence of direct measurements, we assume that the human lens values are representative of the cornea as well for an order of magnitude estimate; the similarity of rabbit corneal epithelium and lens concentrations of NADH support this assumption. It is worth noting that the NADH concentration tends to decrease significantly due to cataracts, so the signal may vary as a result of other eye conditions.

Assuming the cornea has a mass density of 1 kg/m³ (i.e., that of water), the NADH and NADPH number densities are 3×10¹⁶ and 6×10¹⁵ cm⁻³, respectively. The absorption cross-section of NADH at 370 nm is approximately 1.6×10⁻¹⁷ cm², and the FQY is approximately 0.02, resulting in a fluorescence coefficient k_{f} of approximately 1 m⁻¹ (this value is slightly larger at 340 nm excitation). This value is much smaller than that of the representative bacteria and fungi. However, the lens is quite thick and absorbs the majority of UV radiation. The net result is that intrinsic eye fluorescence is likely similar in magnitude as microbial fluorescence for a 0.1 mm thick infiltrate.

For diagnosis of microbial keratitis, for example, intrinsic eye fluorescence is likely large enough that corrections will need to be performed, but still small enough that corrections are reasonably small. These results also suggest that intrinsic eye fluorescence may be used to examine other pathologies of the lens and cornea, for example, the detection and grading of cataracts. Fluorescence bandshape and intensity is also expected to change as a result of chemical and physiological changes in the eye.

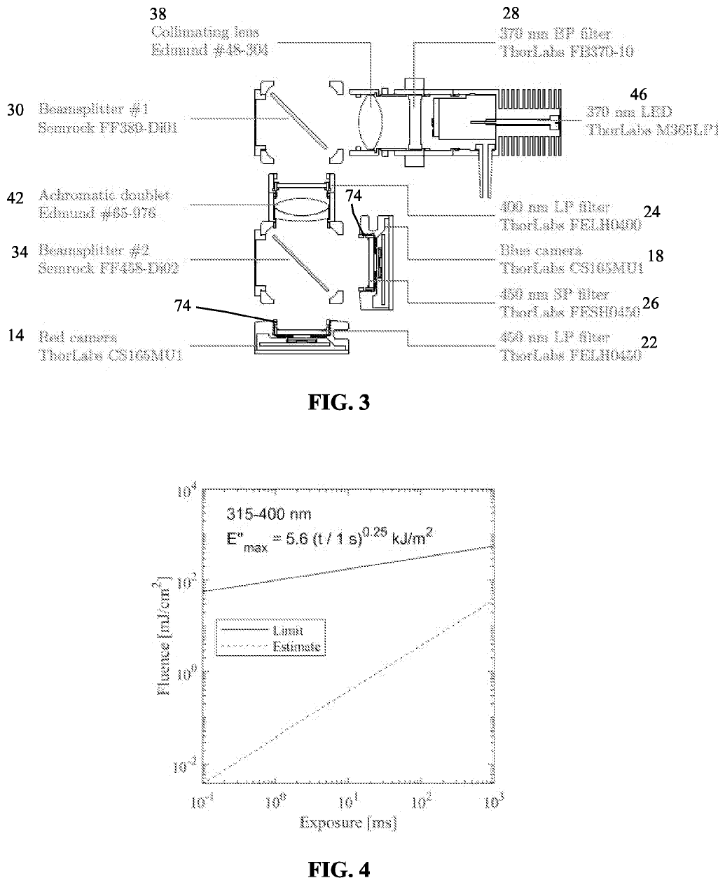

Regarding UV-radiation exposure limits, a significant concern regarding the application of photoluminescence diagnostics to imaging of the human eye is the potential for damage from the ultraviolet excitation. The International Commission on Non-Ionizing Radiation Protection (ICNIRP) releases guidelines on the maximum allowable exposure to nonionizing radiation without causing adverse effects. For an LED source with emission between 315 and 400 nm as proposed here, maximum exposure (fluence) is given as a function of exposure duration t by EQN. 9.

$${E}_{max}^{″} = {A ( \frac{t}{{t}_{0}} )}^{0.25} \qquad EQN . 9$$

Where A=5.6 kJ/m² and t₀=1.0 s is the reference exposure duration. This limit is for a single pulsed exposure, and for t<0.35 s, represents an average over a 1 mm diameter aperture. The exposure limit is plotted as a function of exposure time in FIG. 4, with an estimated exposure superimposed. FIG. 4 illustrates the calculated exposure limits for 370 nm excitation based on ICNIRP guidelines as a function of exposure duration for a single exposure, with an estimated exposure for a 1.5 W LED superimposed. The estimate is calculated assuming a constant optical power of 1.5 W, with 10% collection efficiency focused onto a square region with side length of 2 cm, which is reasonable for the proposed design and corresponds to an incident irradiance of 37.5 mW/cm². From the plot, the estimated exposure is at least an order of magnitude smaller than the limit at every duration considered here. Even with perfect collection efficiency and focusing, the incident fluence would be significantly smaller than the ICNIRP guidelines.

For repetitive exposures, the calculation is unchanged; the same exposure limit applies to each individual pulse in the train, and for the total amount of energy deposited over the duration of the test. However, an additional correction is needed for cases in which retinal heating may be significant. For a given number of pulses, the limiting duty cycle can be calculated by setting the total exposure over time ntₚ equal to the fluence limit over the time interval ntₚ/η where tₚ is the individual pulse duration, n is the number of pulses, and η is the duty cycle. Solving for duty cycle with EQN. 10.

$$η = {t}_{p} {n [ \frac{{E}_{p}^{″} n}{A} ]}^{- 4} \qquad EQN . 10$$

where

$${E}_{p}^{″}$$

is the ruence or exposure rrom a single pulse. This function is plotted in FIG. 5 for several pulse durations as a function of pulse count. FIG. 5 illustrates the maximum allowed duty cycle as a function of pulse count for several exposures at 37.5 mW/cm² incident optical power. From the plot, on the order of one thousand pulses are required at the sample exposures before the duty cycle must be reduced significantly below 1 for exposures on the order of 10 to 100 ms at the assumed irradiance. Assuming a typical exposure duration of 50 ms with a train of 1000 pulses and duty cycle of 0.5, the upper limit on irradiance is approximately 35 mW/cm² and the exposure limit is 1.75 mJ/cm² per pulse. In some embodiments, the device 10 operates under conditions to satisfy the requirements for ISO 15004.2 standard for Group 2 devices.

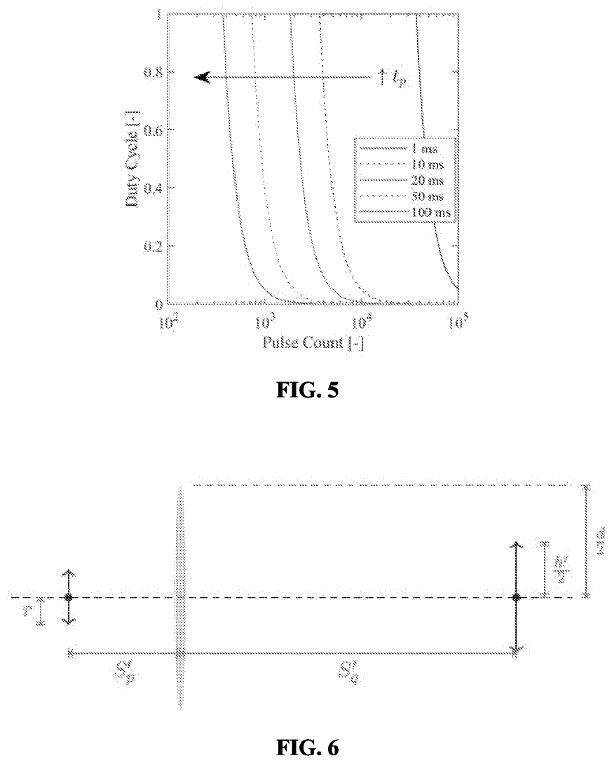

Regarding collimation of a diffuse source, the sources are chosen for this application are diffuse LEDs which are nearly Lambertian surfaces. As a result, the irradiance decays quickly with distance from the source. To achieve good diagnostic performance it is thus critical to gather as much light as possible using a collimating lens and to direct it efficiently onto the cornea. The assumed geometry is illustrated in FIG. 6, which illustrates an optical layout for the LED collimation optics using a lens of focal length f. The black arrows in FIG. 6 indicate the source and target (e.g., cornea) locations. A simple collimation setup would have S′ₚ=f. This would result in all rays from an infinitesimal source (r→0) being perfectly collimated with diameter d. However, since the source has a finite size, rays originating off the optical axis diverge with angle of approximately y/f where −r≤y≤r is the height from which the ray originates. Since the distance S′_{q} is relatively large, this results in a significant divergence of the source. Further, even a perfectly collimated source is distributed over an area larger than the region of interest (determined by the lens diameter), and the collection efficiency depends only on the focal length of the light.

Instead, the source will be slightly focused onto the eye. This allows for a larger collection efficiency since the lens can be moved closer to the source and additional light can be captured and focused onto the target using an increased lens diameter. The ray transfer analysis is relatively straightforward. The intensity at each surface (the source, the front of the lens, the back of the lens, and the target) are a function of height and incidence angle. Assuming no intensity is lost due to reflection or refraction at the boundaries, the intensity at the target surface is identical to that at the source, or as EQN. 11.

$$I ( y , θ ) = {I}_{0} ( {y}_{0} ( y , θ ) , {θ}_{0} ( y , θ ) ) = \{ \begin{matrix}{I}_{0} cos {θ}_{0} & - r \leq {y}_{0} \leq r \\ 0 & otherwise\end{matrix} \qquad EQN . 11$$

Where it is assumed that the source is Lambertian with a fixed radius r. From the expression, we need only to filed the expression for the initial ray height and incidence angle for a given height and angle at the target plane. From geometrical optics, the ray angle and height as the exit of the lens is given by EQN. 12 and EQN. 13

$${θ}^{″} = θ \qquad EQN . 12$$

$${y}^{″} = y - {S}_{q}^{′} tan ( θ ) \qquad EQN . 13$$

Which results from propagating the ray backwards from the target to the lens. Next, the perfectly thin les only redirects the rays and does not alter their height. See EQN. 14 and EQN 15.

$${θ}^{′} = {θ}^{″} + \frac{{y}^{″}}{f} = θ + \frac{y - {S}_{q}^{′} tan ( θ )}{f} \qquad EQN . 14$$

$${y}^{′} = {y}^{″} = y - {S}_{q}^{′} tan ( θ ) \qquad EQN . 15$$

Finally, propagating back to the source plane results in EQN. 16 and EQN. 17.

$${θ}_{0} = {θ}^{′} = θ + \frac{y - {S}_{q}^{′} tan ( θ )}{f} \qquad EQN . 16$$

$${y}_{0} = {y}^{′} - {S}_{p}^{′} tan ( {θ}_{0} ) = y - {S}_{q}^{′} tan ( θ ) - {S}_{p}^{′} tan ( θ + \frac{y - {S}_{q}^{′} tan ( θ )}{f} ) . \qquad EQN . 17$$

Which leads to the result of EQN. 18.

$$I ( y , θ ) = \{ \begin{matrix}\begin{matrix}{I}_{0} cos ( θ + \frac{y - {S}_{q}^{′} tan ( θ )}{f} ) Θ ( r - \\ \vert y - {S}_{q}^{′} tan ( θ ) - {S}_{p}^{′} tan ( θ + \frac{y - {S}_{q}^{′} tan ( θ )}{f} ) \vert )\end{matrix} & {θ}_{-} \leq θ \leq {θ}_{+} \\ 0 & otherwise\end{matrix} . \qquad EQN . 18$$

Where θ_{±} are the upper and lower boundaries on the incidence angles that are collected by the lens and θ is the Heaviside function. The boundaries θ_{±} are determined by the lens geometry according to geometric constraints of EQN. 19.

$$arctan ( \frac{- \frac{d}{2} - y}{{S}_{q}^{′}} ) \leq θ \leq arctan ( \frac{\frac{d}{2} - y}{{S}_{q}^{′}} ) ⟹ {θ}_{\pm } = arctan ( - \frac{\frac{\pm d}{2} - y}{{S}_{q}^{′}} ) \qquad EQN . 19$$

Note that in general, the paraxial approximation is insufficient to model this problem except for small values of d compared to both S′ₚ and S′_{q} or at very small values of y, and thus no further simplification is provided. The incident intensity pattern on the target is calculated as the integral over incident angle provided by EQN. 20.

$$I ( y ) = {{\int }_{{θ}_{-}}}^{{θ}_{+}} d θ I ( y , θ ) \qquad EQN . 20$$

In some embodiments, the integral of EQN. 20 is implemented numerically in computer software (e.g., Matlab) due to the difficulty in solving y₀=±r for θ.

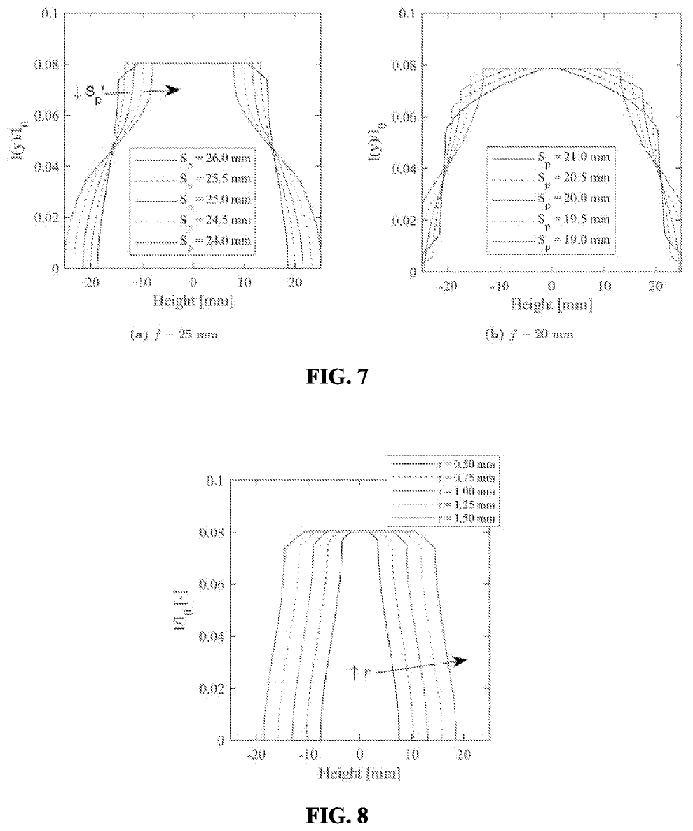

With reference to FIG. 7, sample fluence profiles are calculated using Equation 20 for S′_{q}=300 mm, d=25 mm, and r=1.5 mm, and variable S′ₚ using two different lens focal lengths (Left: f=25 mm; Right: f=20 mm). Several sample fluence profiles calculated with EQN. 20 are shown in FIG. 7 in which the focal length is fixed at f=25 mm, S′_{q}=300 mm, d=25 mm, and r=1.5 mm while S′ₚ is varied within 1 mm off. As seen in FIG. 7, as the source is pulled further behind the lens it is focused tighter onto the target. At S′_{q}=26 mm, 1 mm behind the focal point, the tails of the distribution beyond y of approximately ±15 mm are significantly reduced. However, the peak fluence is fixed and does not change significantly with S′ₚ. From a design standpoint, the LED position is chosen to generate a uniform fluence profile over the region of interest and a sharp cutoff at the edge.

Regarding device focus, due to some of the previously mentioned constraints (e.g., variation in cornea depth between patients, positioning error, patient head motion, fixed image plane distance, etc.), the device is mounted on an adjustment assembly (e.g., a track) that allows position of the device aperture relative to the patient's eye using a motorized stage, for example. In some embodiments, the position of the device aperture relative to the patient's eye is moved along the adjustment assembly manually. The stage is only required to travel on the order of 20 mm. In some embodiments, the stage is motorized so the device can be focused automatically. To facilitate this, a molded rubber eyepiece is mounted to the device such that the object plane is approximately 1 cm beyond the aperture with an adjustable lens tube that allows the eyepiece to travel up to at least 1 cm in either direction. Focus is achieved by translating the system along the optical axis until the location is found that maximizes either the entropy, variance, or sum modulus difference of a fluorescence image of the eye.

In some embodiments, a field stop aperture is used to create a sharp edge in the illumination to provide contrast in the focus images. The effective length of the LED source will be reduced using an aperture to achieve this. To illustrate the effect, the intensity profile is recalculated for sources of varying length using the theory from above and parameters from FIG. 7 (Left), and is plotted in FIG. 8 as a function of the aperture (effective source size) r. In other words, FIG. 8 illustrates calculated fluence profile for several different source lengths and f=25 mm, S′_{q}=300, d=25 mm, and S′ₚ=26 mm. Reducing the source length linearly reduces the width of the uniformly illuminated region, but the shape of the tail is largely unchanged. The configuration chosen here has a relatively steep drop-off in intensity at the edge of the illuminated region which is advantageous for focusing. Note that a field stop aperture cannot be placed near the eye because this would also impact the field of view of the camera. Regarding an alternative approach, there are several drawbacks to using intrinsic eye fluorescence for focusing. Focusing on fluorescence necessarily requires an additional UV exposure which can potentially damage the eye. Intrinsic fluorescence from the eye is also likely to be somewhat diffuse as the cornea and lens are distributed sources, and there may be significant scattering or other radiative transfer of excitation or fluorescence light that can impact the image sharpness.

In some embodiments, an approach is implemented based on diffuse reflection of a broadband blue source from the eye. To achieve this, a low-power blue LED module will be placed in the eyepiece and directed at the eye; the reflected blue light will then be focused onto the blue band camera. The focal point will be determined using the blue camera by finding the location that maximizes entropy, variance, or sum modulus difference of the reflection image.

Turning now to the diagnostic device 10 performance and analysis. A two-camera ratiometric luminescence-based imaging diagnostic is proposed to take advantage of the fluorescence properties and satisfies the constraints identified herein. An overview of the diagnostic operating principle was already provided and now the design requirements are summarized, design decisions are presented, and a detailed analysis of diagnostic performance is provided.

Regarding the summary of requirements and design choices, several design targets or constraints were developed that are summarized in Table 1. In other words, Table 1 illustrates an overview of design constraints with the top portion contains constraints based on diagnostic requirements, while the bottom portion contains constraint derived from the assumed imaging geometry. The imaging parameters (spatial resolution, spatial extent, and depth of field) are related to human physiology and geometry, while the exposure and irradiance limits were chosen to ensure the device is not operated in a way that could damage the patient's eye. Finally, the translation range is necessary to allow focusing of the device as the image plane distance is fixed.

TABLE 1

| Parameter | Constraint | |
|---|---|---|
| Minimum image spatial extent | 18 | mm |
| Minimum image resolution | 5 | mm⁻¹ |
| Minimum depth of field | 5 | mm |
| Maximum exposure | 50 | ms |
| Maximum irradiance | 35 | mW/cm² |
| Minimum translation range | 20 | mm |
| Minimum sensor length | 3.6 | mm |
| Maximum pixel size | 40 | μm |

So far, the geometry of the system is unconstrained such that there are two free imaging parameters: image magnification and lens focal length. As discussed herein, it is advantageous to have a short focal length to maximize light collection (for a fixed aperture), while magnification should be chosen such that the sensor and pixel size are sufficient to satisfy the imaging region and spatial resolution requirements.

Due to optical limitations of a singlet lens, the focal length of the lens must be shorter than the image plane distance Sᵢ, and the magnification of EQN. 21 will generally be on the order of −0.5<M<−0.1.

$$M = - \frac{{S}_{i}}{{S}_{o}} \qquad EQN . 21$$

Thus, for a reasonably compact system with sufficient space for beam splitters and filters, approximately 50<Sᵢ<75 mm, which would require f of approximately 50 mm. As such, the device 10 includes f=50 mm and Sᵢ=60 mm. This provides a sufficient space between the imaging lens and sensor to allow for mounting of the beam splitter, band-pass filter, and provide for any optical adjustment necessary. This additionally results in a relatively low magnification of −0.2 such that small image sensor formants can be used while maintaining the required spatial resolution, greatly reducing the cost of the device. The minimum image sensor size is 3.6 mm, and the maximum effective pixel size is approximately 20 μm. Finally, a lens diameter d of the device 10 is 25 mm such that f_{#}=2.

With these parameters, the object plane distance is 300 mm and the depth of field (DOF) around the object plane is approximately given as EQN. 22.

$$DOF = \vert \frac{2 {S}_{o}}{\frac{2 dM}{{l}_{o}} - \frac{{l}_{o}}{2 dM}} \vert \qquad EQN . 22$$

Such that DOF is approximately 12 mm to match the minimum required spatial resolution (at higher resolutions, the DOF is necessarily more shallow). The diffraction-limited spot size l_{D} is given as l_{D}=1.22λf_{#}—with l_{D} of approximately 1 μm which is well within the required resolution limit. Lens-dependent aberrations will greatly increase the spot size, so these are investigated further for the selected lines.

Regarding camera selection, the device 10 includes low-cost imaging sensors 14, 18. The relevant specifications are provided in Table 2. Each camera uses a monochrome CMOS sensor of a similar size and has a hardware trigger via a general purpose input/output (GPIO) pins.

TABLE 2

| Model | Mfgr. | lᵢ | Size | N_{b} | η_{QE} | N_{FW} | N₀ | $ |
|---|---|---|---|---|---|---|---|---|
| CS165MU1 | ThorLabs | 3.45 | 4.97 × 3.73 | 10 | 0.692 | 11,000 | 4.0 | 530 |
| EO-1312 LE/UI-3240 | Edmund Optics | 5.3 | 6.79 × 5.43 | 10 | 0.62 | 12,000 | 8.5 | 595 |
| BFS-U3-16S2M-CS | FLIR | 3.45 | 4.97 × 3.73 | 10, 12 | 0.648 | 11,200 | 2.5 | 425 |
| Alvium 1800 U-040m | Allied Vision | 6.9 | 5.0 × 3.8 | 8, 10, 12 | 0.64 | 20,800 | 3.2 | 345 |
| ace2 a2A2590-00umBAS | Basler | 2.0 | 5.2 × 3.9 | 8, 10, 12 | 0.723 | 7,400 | 2.0 | 325 |
| | | μm | mm | bits | e⁻/γ | e⁻ | e⁻ | USD |

Table 2 provides a comparison of camera parameters including pixel size lᵢ, sensor size, ADC resolution Nb, quantum efficient η_{QE}, full-well capacity N_{FW}, and read-noise N₀. Read-noise is estimated from full-well capacity, ADC resolution and peak signal-to-noise ratio provided by the manufacturer where not provided directly. Quantum efficiency is measured at 520 nm. As noted in Table 2, the two best performing sensos for the purpose of this diagnostics are the Basler ace2 a2A2590-60umBAS and the ThorLabs CS165MU1, which have the highest quantum efficiencies of those models compared. The performance of the Basler ace2 a2A2590-60umBAS is overall slightly improved over that of the ThorLabs CS165MU1 camera with slightly improved quantum efficiency, lower read noise, higher pixel density, and lower cost. However, in some embodiments, the ThorLabs CS165MUS1 camera is utilized because it is a scientific camera with provided calibration information and does not have a fixed lens mount, which provides more flexibility for opto-mechanic layout. In some embodiments, any one of the camera models of Table 2 is utilized in the device 10. In some embodiments, at least two cameras are utilized in the device. In some embodiments, at least three cameras are utilized in the device.

Regarding imaging lens selection, the imaging lens 42 of the illustrated embodiments has a focal length of 50 mm, be is highly transmissive from 400 nm to at least 550 nm. The imaging lens 42 has minimal monochromatic and chromatic aberrations. There are three options for singlet lens: spherical, aspherical, and achromatic. An aspherical lens is generally a significant improvement over a spherical lens in terms of reduced monochromatic aberrations, but can perform poorly if used at wavelengths significantly different from the design wavelength. An achromatic lens instead is actually two lenses of different material cemented together. The second lens with different material and refractive index is used to correct for chromatic aberrations introduced by the primary lens, but may still suffer from significant monochromatic aberrations.

An aspherical lens (e.g., Edmund Optics #33-945) and an achromatic lens (e.g., Edmund Optics #65-976) were both considered as options for various embodiments, and their point spread functions (PSFs) were determined using a custom raytracer to estimate their performance for this application. The PSF calculation is as follows. A large number of rays are simulated originating from a point source at a distance of 300 mm from the principal plane of the lenses along the optical axis. The rays are uniformly distributed in incidence angle, resulting in an isotropic point source. The ray paths are propagated through the lens system according to Snell's law (ignoring reflection losses) until they reach the image plane, or until they are terminated as a result of total internal reflection. The ray height on the image plane is stored for each ray, which is then used to generate a probability density function (PDF), which is a direct measurement of the PSF. For each simulation, the image and object distances are fixed at 60 mm and 300 mm from the principal planes of the lenses (as provided by the manufacturer).

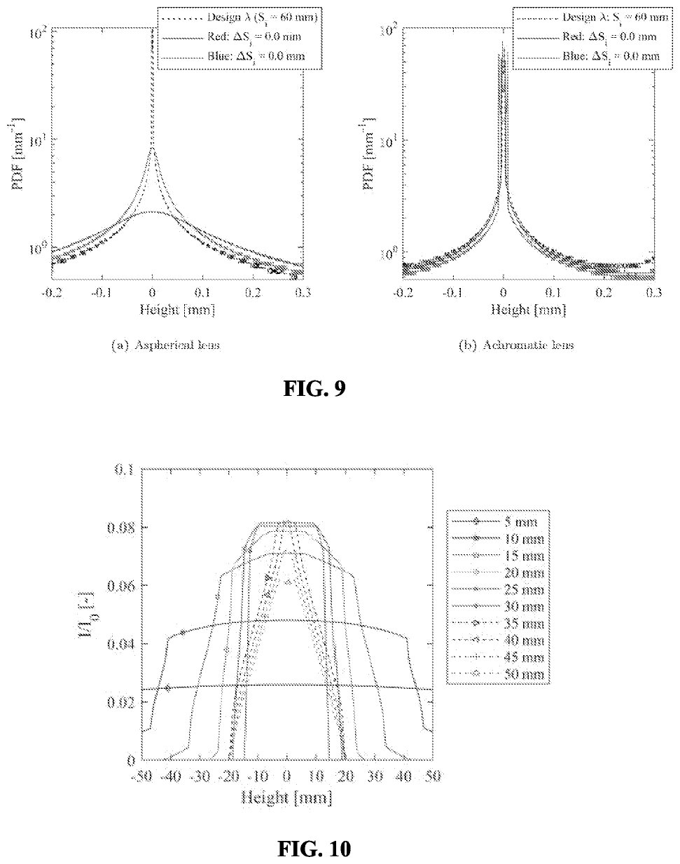

The measured PSFs for the achromatic and aspheric lenses at the design wavelength and at the red and blue imaging band wavelengths are shown in FIG. 9. In other words, FIG. 9 illustrates the calculated PSFs for the achromatic (right, e.g., Edmunds Optic #65-976) and aspherical (left; e.g., Edmund Optics #33-945) lenses at several wavelengths. For the calculation, the image plane and object plane distances are fixed at 60 mm and 300 mm from the principal plane, respectively. With reference to FIG. 9, the full-widths of the achromatic lens are 7, 9, and 19 μm for the design wavelength (405 nm), the blue band, and the red band, respectively. At the design magnification of −0.2, the object plane width is less than 0.1 mm at worst, which is within the required resolution. The aspherical lens clearly has much more significant chromatic aberration. However, in the best case the PSF width is less than 4 μm for wavelengths in focus (using the design wavelength calculation for comparison).

It is worth noting that the change in the PSF of the singlet lenses is a function primarily of the refractive index, which changes the focal length of the lens. Thus, the measured PSF is actually the result of a defocus aberration. By adjusting the image plane location accordingly (e.g., by moving the sensor slightly closer to or further from the lens), the PSF of the lens is constant to a very good approximation and instead the image magnification is changed. Using the raytracer, the relative shift in the image distance for the aspheric lens is −0.4 mm for the red band (530 nm) and −1.2 mm for the blue band (450 nm), resulting in a difference in magnification of ˜1.5% between the red and blue bands. So, resolution of ˜20 μm is possible using the aspherical lens, but with 1.5% change in magnification between the two bands. Since the achromatic lens has sufficient imaging resolution for each band at constant magnification, the achromatic lens is chosen instead in some embodiments. In other embodiments, the aspherical lens is utilized in case it is discovered that the chosen 5 mm⁻¹ resolution is insufficient.

Regarding collimating lens selection, a second lens 38 is needed for collimation of the diffuse LED source 46. A diffuse source can be collimated by placing a converging lens with focal length f′ at a distance of S′ₚ=f′ from the source. Diffuse sources will diverge from this configuration due to their finite width with an angle of θ_{d}≈r/f′ where r is the radius of the source. Since the imaging lens requires a large object plane distance, the excitation light must also propagate a significant distance and may diverge significantly. Instead, the LED will directed onto the cornea, and the imaging parameters will be chosen to maximize fluence.

The distance from the lens to the cornea is fixed at S′_{q}=Sₒ≈300 mm. The lens focal length f′ and distance from lens to source S′ₚ must be determined. The lens diameter dis 25 mm to match the aperture used for the imaging lens, the intended illumination height is h′≈20 mm, and the source radius r is approximately 1.5 mm. Refer to FIG. 6 for an illustration of the lens geometry and symbol definitions.

For a given focal length, the lens to source distance S′ₚ should be reasonably close to f′ (to be nearly collimated), but is adjusted slightly to more tightly focus the beam. For simplicity, we choose the optimal distance S′ₚ as the distance where the kurtosis of the distribution is minimized (corresponding to the configuration where the amount of energy in the tails is minimized), as provided in EQN. 23.

$${S}_{p , opt}^{′} = {arg min}_{{S}_{p}^{′}} \frac{\int I ( y ; {S}_{p}^{′} , {S}_{q}^{′} , r , d , f ) dy \int I ( y ; {S}_{p}^{′} , {S}_{q}^{′} , r , d , f ) {y}^{4} dy}{{( \int I ( y ; {S}_{p}^{′} , {S}_{q}^{′} , r , d , f ) {y}^{2} dy )}^{2}} . \qquad EQN . 23$$

In some embodiments, EQN. 23 is evaluated numerically in computer simulation (e.g., Matlab) for a variety of lens focal lengths. The resulting profiles, I (y; S′_{p,opt}, S′_{q}, r, d, f), are plotted in FIG. 10. Notably, there is a tradeoff between collection efficiency and beam divergence that is evident as focal length is varied from 5 to 50 mm. At very short focal lengths the collection efficiency is high so much of the emission is captured, but due to the high divergence associated with short focal lengths, the intensity in the region of interest is actually relatively low. Conversely, the 50 mm focal length has very narrow divergence such that the beam exactly covers a relatively small area, but the peak intensity is low due to the poor collection efficiency. The peak fluence in the region of interest actually occurs for the intermediate 35-30 mm focal length lens and the uniform region matches the region of interest at approximately 20 mm wide.

In some embodiments, the device 10 includes an Edmund Optics #48-304 double convex UV anti-reflection coated lens with 25 mm focal length and 25 mm diameter. From the calculation, the lens is placed at S′ₚ=26.4 mm from the source for an object plane distance of 300 mm.

Regarding device layout, materials, and cost; the mechanical layout of the device 10 uses a cage system (e.g., ThorLabs 30 mm) in some embodiments. In some embodiments, the thin-film interference filters, the LED, and the cameras are also from ThorLabs. In some embodiments, the imaging and collimating lenses are from Edmund Optics and the beam splitters are from Semrock, Inc. In some embodiments, the device 10 includes computer control provided by a processor 12 (e.g., a Raspberry Pi) that provides on-board graphics processing capabilities for image analysis, direct camera control of USB using python and C++ software development kits (SDKs) provided by ThorLabs. In some embodiments, the processor 12 is integrated within the device 10.

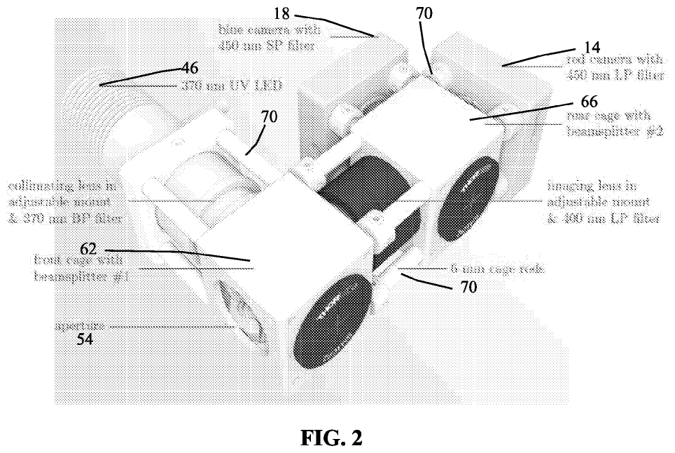

With reference to FIG. 2, an embodiment of the device 10 is illustrated. The device 10 includes two beam splitters that are each housed in a cage 62, 66 (e.g., a 33 mm cage). The front cage 52 holds the beam splitter 30 that reflects UV light to the side and transmits visible light along the optical axis 58. To the side of the front cage 62 (following the path of UV light), the UV LED 46 is mounted with the collimating lens 38 and the bandpass filter 28 (e.g., a 370 nm bandpass filter). Immediately behind the front cage 62 along the optical axis 58 is the rear cage 66, which is mounted using 6 mm cage rods 70. The rear cage 66 contains the visible beam splitter 34. In some embodiments, the beam splitter 34 includes a cutoff wavelength of approximately 458 nm. An exit aperture of the front cage 62 contains the imaging lens 42 and a 400 nm long-pass filter 24 in an adjustable mount.

With continued reference to FIG. 2, the imaging camera 18 (e.g., a blue imaging camera) is placed at a side aperture of the rear cage 66 and the imaging camera 14 (e.g., a red imaging camera) is placed at a rear aperture of the rear cage 66. In the illustrated embodiment the imaging cameras 14, 18 are mounted to the rear cage 66 with cage rods 70 (e.g., 6 mm cage rods). In some embodiments embodiment, the cameras 14, 18 have a 450 nm long-pass filter or and a 450 nm short-pass filter mounted directly in threaded apertures 74 of the ThorLabs SM1 (See FIG. 3).

In some embodiments, the device 10 includes a frame 50 including an aperture 54 defining an aperture axis 58 and an imaging lens 42 aligned with the aperture axis 58. The device 10 includes a first image sensor 14 with a first imaging axis 58 aligned with the aperture axis 58, and a second image sensor 18 with a second imaging axis 57. The device 10 further includes a light source 46 configured to emit a light along a source axis 59. A first beam splitter 30 is positioned at an intersection of the aperture axis 58 and the source axis 59, and a second beam splitter 34 is positioned at an intersection of the first imaging axis 58 and the second imaging axis 57. In the illustrated embodiment, the source axis 59 is orthogonal to the aperture axis 58, and the source axis 59 is parallel to the second imaging axis 57. In the illustrated embodiment, the second imaging axis 57 is orthogonal to the first imaging axis 58. In some embodiments, the device 10 is a hand-held line-of-sight diagnostic tool.

In some embodiments, the device 10 further includes a first filter 22 aligned with the first image sensor 14, a second filter 26 aligned with the second image sensor 18; and a third filter 38 aligned with the source axis 59.

In some embodiments, the device 10 further includes a collimating lens 38 aligned with the source axis 59. In some embodiments, the collimating lens 38 is positioned between the third filter 28 and the light source 46. In some embodiments, the device 10 further includes a variable aperture positioned behind the collimating lens 38.

In some embodiments, the device 10 includes a cutoff filter positioned between the imaging lens 42 and the first beam splitter 30.

In some embodiments, the imaging lens 42 is an achromatic lens, a spherical lens, or an aspherical lens. In the illustrated embodiment, the imaging lens 42 is positioned between the first beam splitter 30 and the second beam splitter 34 along the aperture axis 58. In some embodiments, the imaging lens 42 has a focal length of 50 mm. In some embodiments, the imaging lens 42 is transmissive within a range of approximately 400 nm to approximately 700 nm. In some embodiments, the camera is sensitive to approximately 1100 nm.

In some embodiments, the first filter 22 is a 450 nm long-pass filter, and the second filter 26 is a 450 nm short-pass filter. In some embodiments, the first filter 22 is positioned within a threaded aperture 74 of the first image sensor 14, and the second filter 26 is positioned with a threaded aperture 74 of the second image sensor 18.

In the illustrated embodiment, the second beam splitter 30 has a cutoff wavelength of approximately 458 nm. In some embodiments, the light source 46 is a light emitting diode (LED). In other embodiments, the light source 46 is a laser diode or a discharge lamp (e.g., a Mercury discharge lamp). In some embodiment, the light source 46 is an ultraviolet light emitting diode.

In the illustrated embodiment, the frame 50 includes a first cage 62, a second cage 66, a plurality of cage rods 70 extending from the first cage 62 and the second cage 66. The first beam splitter 30 is positioned within the first cage 62 and the second beam splitter 34 is positioned within the second cage 66. In some embodiments, the first image sensor 14 and the second image sensor 18 are coupled to the second cage 66, and the light source 46 is coupled to the first cage 62. In the illustrated embodiment, the imaging lens 42 is positioned between the first cage 62 and the second cage 66. In some embodiments, a distance between the imaging lens 42 and the first image sensor 14 is approximately 60 mm.

In the illustrated embodiment, the image plane distance is 60 mm, and the collimating lens to source distance is 26.4 mm. Both distances are adjustable using the adjustable lens mounts. The image plane distances of each camera are also slightly adjustable using the 6 mm rods to correct for wavelength-dependence in the imaging lens focal length if desired.

Materials for one embodiment of the device 10 are listed in Table 3. In one embodiment, the ThorLabs 30 mm cage system was chosen for the majority of the optomechanics, the LED, cameras, and filters. In some embodiments, the device includes a custom designed optomechanic platform that has reduced cost, more precise optical component layout, and reduced geometric constraints that make it easier to incorporate other hardware such as the Basler ace2 camera.

TABLE 3

| Item | Manufacturer | Part No. | Quantity | Each | Total |
|---|---|---|---|---|---|
| Rectangular filter cage cube, 30 mm | ThorLabs | CM1-DCH | 2 | 175.31 | 350.62 |
| 30 mm cage system rod, 0.25″ | ThorLabs | ER025 | 3 | 5.20 | 41.60 |
| 30 mm cage system rod, 1.0″ | ThorLabs | ER1 | 4 | 5.20 | 20.80 |
| 30 mm cage system rod, 1.5″ | ThorLabs | ER15 | 4 | 6.03 | 24.12 |
| 30 mm cage system rod adapter | ThorLabs | ERSCB | 12 | 15.00 | 180.00 |
| 30 mm cage plate, 1.2″ dia. bore | ThorLabs | CP36 | 1 | 22.07 | 22.07 |
| 1″ lens tube, 0.3″ | ThorLabs | SM1L03 | 1 | 12.52 | 12.52 |
| 1″ lens tube, 0.5″ | ThorLabs | SM1L05 | 3 | 12.97 | 38.91 |
| 1″ lens tube, adjustable 0.5″ | ThorLabs | SM1V05 | 1 | 31.16 | 31.16 |
| 1″ lens tube end cap | ThorLabs | SM1L03 | 2 | 18.83 | 37.66 |
| 400 nm longpass filter | ThorLabs | FELH0400 | 1 | 131.00 | 131.00 |
| 450 nm longpass filter | ThorLabs | FELH0450 | 1 | 131.00 | 131.00 |
| 450 nm shortpass filter | ThorLabs | FESH0450 | 1 | 131.00 | 131.00 |
| 370 nm bandpass filter | ThorLabs | FB370-10 | 1 | 145.08 | 146.08 |
| 1.5 MP CMOS camera | ThorLabs | CS165MU1 | 2 | 535.00 | 1070.00 |
| 365 nm mounted LED, 2000 mW | ThorLabs | M365LP1 | 1 | 460.99 | 460.99 |
| LED power supply | ThorLabs | LEDD1B | 1 | 323.55 | 323.55 |
| 389 nm dichroic beamsplitter, 25 × 36 mm | Semrock, Inc. | FF389-D101-25 × 36 × 1.5 | 1 | 355.00 | 355.00 |
| 453 nm dichroic beamsplitter, 25 × 36 mm | Semrock, Inc. | FP458-D102-25 × 36 × 1.5 | 1 | 285.00 | 285.00 |
| 25 mm dia, 25 mm f.L., UV-vis coated | Edmund Optics | #48-304 | 1 | 133.00 | 133.00 |
| spherical lens | | | | | |
| 25 mm dia, 50 mm f.L., UV-vis coated | Edmund Optics | #65-976 | 1 | 134.50 | 134.50 |
| achromatic lens | | | | | |
| Single board computer | Raspberry P1 | Pi 4 B | 1 | 50.00 | |
| Total | | | | | 4110.68 |

In some embodiments, a system for fluorescence-based corneal infection imaging is provided and includes an aperture 54 configured to be placed in proximity to an eye of a patient of subject, a first image sensor 14 configured to capture a first image of the eye, and a second image sensor 18 configured to capture a second image of the eye. The system includes a single imaging lens 42 positioned between the aperture 54 and the first image sensor 14, and a light source 46 configured to provide an excitation light to the eye. In some embodiments, the system further includes a first beam splitter 30 and a second beam splitter 34. In the illustrated embodiment, the system further includes a first filter 22 aligned with the first image sensor 14, a second filter 26 aligned with the second image sensor 18, a third filter 28 aligned with the light source 46.

In some embodiments, a system for fluorescence-based imaging is provided. The system is used in some embodiments for general fluorescence redox imaging of a biological target. In some embodiments, the biological target is an eye (e.g., lens, cornea, sclera, and anterior segment), a skin sample (e.g., wounds), a cell sample (microbe cultures, smears), or other similar biological targets.

In some embodiment, the system includes a processor 12 in electrical communication with the image sensors 14, 18. In some embodiments, the processor 12 is configured to analyze the first image and the second image to determine the presence of infection in the eye. In some embodiments, the processor 12 is integrated within the device itself. In some embodiment, the system further includes a display 16 configured to display a result of the processor 12 analysis. In some embodiments, the display 16 is integrated with the device itself.

In some embodiments, the system further includes adjustment assembly (e.g., track) to adjust the position of the aperture 54 relative to the eye. In some embodiments, adjusting the position of the aperture 54 relative to the eye includes the distance to the eye, the relative angle to the eye, or any combination thereof. In some embodiments, the system includes an adjustable eyepiece coupled to the aperture. In some embodiments, the position of the aperture 54 relative to the eye is adjusted by energizing a motorized stage.

In some embodiments, the system further includes timing electronics independent of the computer or processor that controls the camera. In some embodiments, the system further includes a controller and pulser for the light source. In some embodiments, the system further includes an optical sensor (e.g., photodiode) or other sensor to monitor the output of the light source. In some embodiments, the system further includes a plurality of calibration targets to assess the device performance and to generate data for making corrections (e.g., a LED profile).

Regarding imaging precision, detection limits, and ratio precision; a ratio measurement R is formed at each pixel as the radio of two background-subtracted fluorescence emission signals, R=S₂/S₁. The fluorescence signal is excited by application of pulsed ultraviolet light at 370 nm, in some embodiments, from a LED which is focused onto the cornea. Emission from the cornea is imaged onto the two image sensors 14, 18 which have identical object plane pixel sizes lₒ. In some embodiments, the fluorescence excitation is assumed linear, and the depth of the infected region is assumed smaller than the depth of field.

The photon flux incident on the cornea is Φ″=E″/hω where hω=5.4×10⁻¹⁹ J and E″ is the local radiant exposure. Within a single image pixel of area

$${l}_{o}^{2} ,$$

there is an assumed microbe volume of

$${l}_{o}^{2} {t}^{′} χ$$

where t′ is the thickness of the microbe layer and χ is the microbe volume fraction (collectively t=t′χ is the effective thickness of the microbe layer). The number of photons emitted from within each pixel volume is then given as EQN. 24.

$$I = {k}_{f} {Φ}^{″} {l}_{o}^{2} t \qquad EQN . 24$$

Of the photons emitted isotropically, only a fraction are collected and converted to signal of EQN. 25.

$$S = I \frac{Ω}{4 π} \frac{{η}_{opt} η QE}{{C}_{AD}} = {k}_{f} {Φ}^{″} {l}_{o}^{2} t \frac{Ω}{4 π} \frac{{η}_{opt} η QE}{{C}_{AD}} \qquad EQN . 25$$

where Ω is the lens collection solid angle, ηₒₚₜ is the optical efficiency of all transmissive components, ηQE is the sensor quantum efficiency, and C_{AD} is the sensor analog-to-digital gain. The final signal Sis expressed in units of counts or ADU. The subscripts indicating the signal collection bands were dropped for brevity.

The sensor noise is assumed to be dominated by thermal noise sources and shot noise, such that EQN. 26.

$${s}_{S}^{2} = S + {( \frac{{N}_{0}}{{C}_{AD}} )}^{2} + \frac{1}{12} + B \qquad EQN . 26$$

where N₀ is the read noise (in electrons), B is the background signal in counts (including any dark signal), and the constant term (1/12) is the quantization noise. The signal-to-noise ratio of a fluorescence signal measurement per pixel is expressed as EQN. 27, and the uncertainty in the ratio measurement is given by EQN. 28.

$${SNR}_{i} = \frac{{S}_{i}}{{s}_{{S}_{i}}} = {\sqrt{{S}_{i}} [ 1 + \frac{1}{{S}_{i}} ( \frac{{N}_{0}^{2}}{{C}_{AD}^{2}} + \frac{1}{12} + B ) ]}^{- 1 / 2} \qquad EQN . 27$$

$$\frac{{s}_{R}^{2}}{{R}^{2}} = \frac{{s}_{{S}_{1}}^{2}}{{S}_{2}^{2}} + \frac{{s}_{{S}_{2}}^{2}}{{S}_{2}^{2}} = \frac{1}{{S}_{2}} ( 1 + R ) + \frac{1}{{S}_{2}^{2}} [ ( \frac{{N}_{0}^{2}}{{C}_{AD}^{2}} + \frac{1}{12} ) ( 1 + {R}^{2} ) + {B}_{2} ( 1 + \frac{{R}^{2}}{{R}_{B}} ) ] \qquad EQN . 28$$

where B₂ is the background signal in the red band and R_{B}=B₂/B₁ is the background ratio.

The ratio SNR (SNR_{R}=R/s_{R}) was calculated as a function of t using the design parameters provided herein for the assumed exposure duration of 50 ms. The excitation irradiance is taken to be the target value of 35 mW/cm2 and an intermediate fluorescence coefficient of 0.1 mm⁻¹ is chosen. The sensor is software binned up to the largest possible pixel size that meets the required maximum length of 100 μm. The optical parameters used in the performance calculations are given in Table 4. The emission spectrum used to calculate quantum efficiency and optical efficiency is that of E. coli, and the fluorescence coefficient is an order of magnitude estimate based on the 340 nm excitation data presented herein. Beam splitter transmission is included in the calculated optical efficiency. To estimate the background contribution, the collection efficiency for each band is chosen to be equal to that for the E. coli luminescence resulting in R_{B}=R.

TABLE 4

| Parameter | Symbol | Value | Units |
|---|---|---|---|
| Fluorescence coefficient | k_{f} | 0\|.1 | mm⁻¹ |
| Excitation irradiance | E″ | 35 | mW/cm² |
| Physical pixel size | lᵢ | 3.45 | μm |
| Effective pixel size | lₒ | 69 | μm |
| Bin value | b | 4 | — |
| Magnification | \|M\| | 0.2 | — |
| Lens f-number | f_{#} | 2 | — |
| Collection fraction | Ω/4π | 4.3 × 10⁻⁴ | — |
| Read noise | N₀ | 4.0 | e⁻ |
| ADC gain | C_{AD} | 10.74 | e⁻/ADU |
| Optical efficiency (red) | η_{opt, 2} | 0.57 | — |
| Optical efficiency (blue) | η_{opt, 1} | 0.23 | — |
| Quantum efficiency (red) | η_{QE, 2} | 0.68 | — |
| Quantum efficiency (blue) | η_{QE, 1} | 0.60 | — |

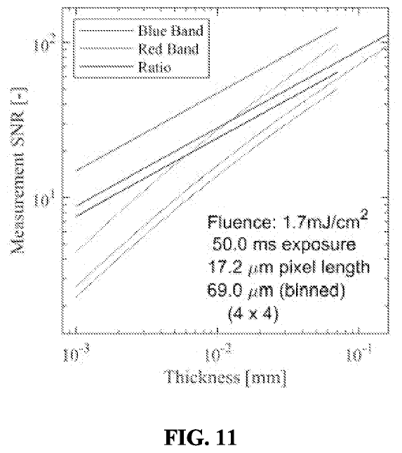

With reference to FIG. 11, the SNR is plotted including (dashed lines) and excluding (solid lines) the effect of background luminescence which is discussed further herein. Single-shot per pixel ratio SNRs are on the outer of 10 for a reasonable thickness of 10 μm, but the red band fluorescence image SNR is somewhat higher. On a per-pixel basis, this may be insufficient to diagnose an infection. However, since the ratio is expected to be uniform throughout the infected region, the ratio can be averaged over the infected region to greatly improve the SNR. A factor of ˜10 improvement in SNR may be sufficient for reliable identification (≤1% uncertainty in the ratio), which would require at least 100 pixels over which an average can be calculated, or an infected region that is at least on the order of 800 μm in diameter.

The image SNR can further be improved somewhat using spatial filtering and temporal averaging. Since the effective pixel size is only ˜70 μm (70% of the maximum allowable size based on the image resolution requirement), some smoothing can be used to reduce noise at the cost of this additional resolution. Measurement uncertainty is expected to scale inversely proportional to spatial resolution, so this can provide approximately 30% improvement in SNR. Additionally, many images (up to 1000) can be taken at up to a 50% duty cycle while maintaining a sufficiently low exposure. Averaging over, e.g., 10 measurements would further improve the precision by a factor of ˜3. For a 50% duty cycle and the specified exposure settings, images can be taken at up to 10 Hz for up to 100 s continuously.

A significant improvement in collection efficiency could be made in the production version of the device by incorporating either a larger imaging lens and aperture, or by reducing the imaging lens focal length (i.e., reducing the lens f_{#}), albeit at the cost of reduced depth of field. The detection limits can be estimated as the thickness at which the SNR is equal to unity. From FIG. 11, this occurs at t=3 μm.

Regarding intrinsic fluorescence from eye tissues, the fluorescence is assumed to be constant and approximately uniform for an individual (neglecting extinction by infecting microbes).

The front of the eye can be analyzed in three parts: the thin outer layer (the cornea), an intermediate transparent layer (the aqueous humour), and the crystalline lens. The cornea strongly absorbs ultraviolet light, such that ˜50% of light at 370 nm is attenuated, and fluoresces due to the presence of pyridine nucleotides and flavoproteins, similar to the microbe samples tested here. Pyridine nucleotides are believed to be the only significant source of fluorescence in the cornea near 450 nm.

After passing through the cornea, the remaining ˜50% of the incident radiation passes through the aqueous humour. It is assumed that fluorescence from the aqueous humour at 370 nm excitation is negligible as the aqueous humour is highly transmissive at near-UV wavelengths and the observed fluorescence peaks at 350 nm, which suggests that 370 nm is inefficient for excitation. The crystalline lens absorbs the vast majority of the remaining UV light, but can fluoresce and likely has a fluorescence coefficient similar to that of the cornea. The lens is also assumed to have a radiant exposure of 50% of the incident exposure due to UV extinction in the cornea. The total eye fluorescence intensity including the contribution of the lens is thus estimated to be 50% larger than that of the cornea alone.

The ultraviolet radiant exposure of the cornea is assumed to be that of the infecting microbes; any extinction due to the microbial film is neglected. The fluorescence coefficient of the cornea was estimated to be approximately 1% of that of the microbial layer, but the cornea depth is constant at approximately 0.5 mm. The signal-to-background ratio then depends only the ratio of the product of the fluorescence coefficient and layer thickness, see EQN. 29.

$$SBR \approx \frac{{k}_{f} t}{3 / 2 {k}_{c} {t}_{c}} \approx \frac{t}{7.5 μm} \qquad EQN . 29$$

where k_{c} and t_{c} are the fluorescence coefficient and thickness of the cornea, respectively. Thus, for a 150 μm-thick microbe layer, the SBR is approximately 20. The fluorescence signal from the cornea is thus significant, but can be corrected for a reasonably thick microbe layer. For smaller layers, the intrinsic eye fluorescence may become sufficiently large that the microbe layer cannot be distinguished. A lower limit on layer thickness is when the SBR is equal to 2, or t=15 μm.

The ratio precision can be rewritten in terms of the SBR if the collection efficiency of the background signal is the same as that of the microbe luminescence, or R_{B}=R. See EQN. 30 In this case such that shot-noise scales with \sqrt{1+SBR⁻¹}.

$$\frac{{s}_{R}^{2}}{{R}^{2}} = \frac{1}{{S}_{2}} ( 1 + R ) ( 1 + {SBR}^{- 1} ) + \frac{1}{{S}_{2}^{2}} ( \frac{{N}_{0}^{2}}{{C}_{AD}^{2}} + \frac{1}{12} ) ( 1 + {R}^{2} ) \qquad EQN . 30$$

Regarding imaging resolution and aberrations, imaging resolution and the impact of aberrations are estimated herein by the shape or width of the PSF for the selected lens when polychromatic light is incident from a point off the optical axis (producing, e.g., coma or third-order astigmatism aberrations) and points along the optical axis away from the object location (producing defocus aberrations, which are related to depth of field). These aberrations can be monochoromatic or polychromatic; in the most general case both types are present. PSFs are calculated for a variety of physically-reasonable imaging conditions using the same raytracing method as described previously, with slight alterations to the input system to identify optical aberrations.

The raytracing procedure is as follows. A sample E. coli emission spectrum weighted by optical transmission and camera quantum efficiency is sampled for the red and blue bands at several discrete representative points. Then, for each wavelength a range of initial source locations is chosen either spanning along the optical axis (axial) or normal to the optical axis (lateral), and rays are generated isotropically that cover the image aperture. Each ray is propagated through the system until it is terminated at the image plane or undergoes total internal reflection. At the image plane, PDFs are generated of the calculated ray height for each sampled wavelength and source location to investigate both chromatic and monochromatic aberrations. The PDF generated for each source height is then shifted such that the median value is zero, resulting in the aberrated PSF. For simplicity, a fixed number of incidence angles are used at each point; this procedure artificially increases the contribution of off-axis sources such that the contribution of lateral aberrations (and to a lesser extent axial aberrations, but not chromatic aberrations) are somewhat exaggerated. This is acceptable here since we're interested only in identifying the worst-case imaging resolution.

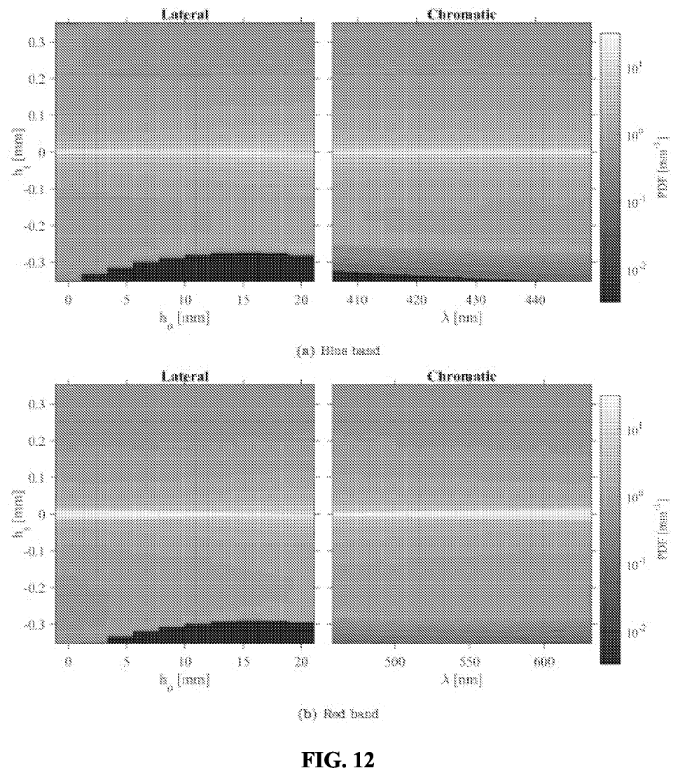

Several PSFs representing lateral aberrations are shown for each imaging band in FIG. 12. First, the lateral aberration plot shows the PSFs averaged over wavelength as a function of source height, while the chromatic aberration plot shows PSFs averaged over all source heights as a function of wavelength. Source object plane heights hₒ were taken up to 20 mm to match the approximate region of interest of the imaging diagnostic. For the calculation, 2000 rays were simulated with uniformly distributed angles of incidence at 10 different object heights and 20 wavelengths. From the figure, aberrations are least significant for the blue band, which primarily images wavelengths near the lens design wavelength. Chromatic aberrations appear to have only a small impact, but lateral aberrations are more significant particularly near the edge of the region of interest. In contrast, the red band image is formed more sharply at the outer edge of the region of interest, but chromatic aberrations are much more significant and sources near the center of the image are more significantly distorted. The PSF tail is asymmetric in all cases except at hₒ=0 mm, but the magnitude is on the order of only a few percent of the peak value.

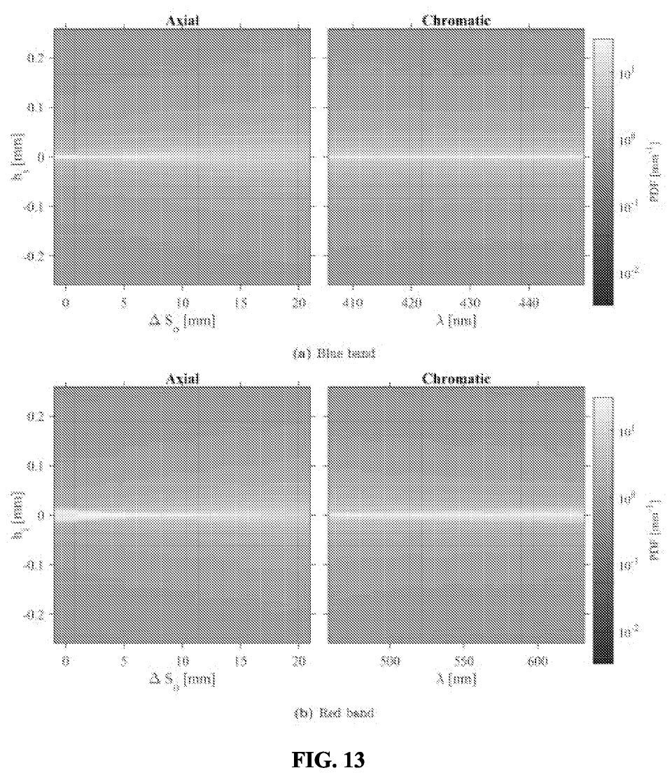

The impact of axial chromatic aberrations were calculated in the same way except the source location was varied along the optical axis relative to the design object distance Sₒ. The axial and chromatic PSFs were calculated identically to the lateral and chromatic PSFs and are shown in FIG. 13. There are several interesting features to note in the plots. First, the camera is actually focused for both bands somewhat further than 300 mm from the principal plane; this is expected due to differences in focal length between the emission wavelengths and the design wavelength. The object plane is actually located at approximately 305-310 mm from the lens principal plane on average. The depth of field is verified to be approximately 10 mm, which corresponds to the range over which the full-width at half-maximum (FWHM) of the PSF is less than 0.1 mm. As before, chromatic aberrations are not significant for the blue band, but are somewhat more so for the red band when averaged over the 20 mm window.

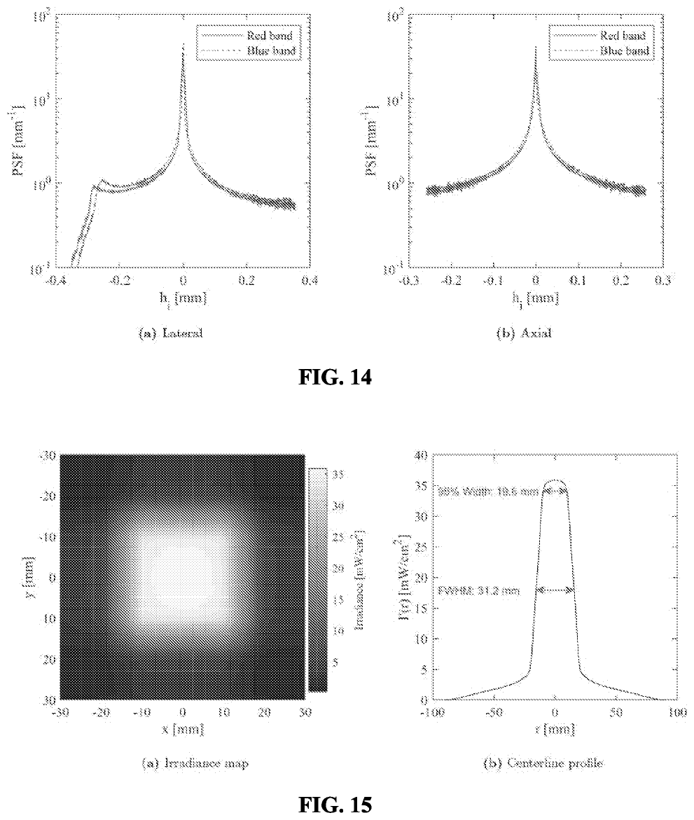

The total PSF averaged over both chromatic and axial or lateral aberrations is plotted in FIG. 14 for each band. Since there is no sharp transition between the peak and baseline, the resolution is estimated from the FWHM of the PSF. The lateral PSFs in FIG. 14a are wider than the axial PSFs and are used to estimate resolution as 9 and 5 μm for the red and blue imaging bands, respectively. Including a factor of two as a conservative limit since FWHM is likely under estimating dispersion, the estimated image plane resolution is still sufficient to achieve the required resolution including expected axial, lateral, and chromatic aberrations.

Regarding radiometric characterization of source, a more robust radiometric analysis of the LED irradiance at the cornea plane is made using the same raytracing method using the collimating lens parameters provided by the manufacturer.

For an absolute irradiance calculation, a fixed number of rays were simulated from each point on the LED surface with incidence angles distributed according to Lambert's cosine law; the incidence angle cumulative distribution function (CDF) for a Lambertian emitter was sampled uniformly and the incidence angle for each CDF value was determined according to CDF=sin(θ)/2. The 2D intensity distribution function I′(r) at the cornea plane is determined using a kernel smoothing estimation technique with a normal smoothing function (e.g., ksdensity in Matlab). The irradiance distribution (power per unit area, I″) is assumed to be identical in the horizontal and vertical directions, as EQN. 31.

$${I}^{″} ( x , y ) = {I}^{′} ( x ) \times {I}^{′} ( y ) \qquad EQN . 31$$

Where I′(r) is the kernel generated from the 2D raytracing simulation (power per unit length). The integral of I″ over x and y is equal to the amount of power collected by the lens given as EQN. 32.

$$\int \int {I}^{″} ( x , y ) dx dy = η {I}_{tot} \qquad EQN . 32$$

where η is the fraction of light collected by the lens, and Iₜₒₜ is the total power emitted by the LED. The collection fraction η is given by the fraction of simulated rays that pass through the lens and reach the cornea plane squared (this accounts for the 3D nature of the problem); this value is approximately 25%. The irradiance map calculated at the cornea plane and the centerline profile are shown in FIG. 15.

The collimation lens collection efficiency is approximately 25% at the selected source and target positions, and the power is distributed approximately over a 20 mm square. There are a few differences from the predictions herein, largely due to the initial inaccurate assumption of a perfect lens; primarily, the edge of the beam is not nearly as sharp as predicted herein likely due to thick lens effects. The peak irradiance in this configuration exactly matches the irradiance limit of 35 mW/cm² determined herein for a 2 W total emission. For this calculation, the LED is placed exactly 20 mm from the front vertex of the lens, and the cornea plane is 290 mm behind the rear vertex.

The present disclosure provides, a method of fluorescence-based imaging of a biological target (e.g., an eye, a skin sample, a cell sample, etc). In some embodiments, the method is for detecting corneal infection including providing the device 10 with the aperture 54 configured to be placed in proximity to an eye of a patient or subject, the light source 46, the first image sensor 14, and the second image sensor 18. The method further includes illuminating the eye with an excitation light from the light source 46. In some embodiments, the excitation light includes approximately 370 nm excitation. The method also includes collecting a first image of the eye with the first image sensor 14 and collecting a second image of the eye with the second image sensor 18. The method further includes analyzing the first image and the second image as described herein to determine whether the eye has an infection. In some embodiments, the method includes analyzing images from at least three image sensors to determine whether the eye has an infection.

In some embodiments, analyzing the first image and the second image includes identifying structures of the eye and identifying boundaries between ulcerated regions of the eye and healthy regions of the eye. In some embodiments, analyzing further includes classifying ulcerated regions of the eye as fungal, bacterial, or uninfected. In some embodiments, analyzing further includes classifying a species of fungus or bacteria present in the eye. In some embodiments, analyzing further includes determining size, shape, location, and severity of the ulcerated regions of the eye. In some embodiments, analyzing further includes classifying the ulcerated regions according to size, shape, location, or pathogen. In some embodiments, the method further includes determining a diagnosis based on the analysis of the first image and the second image and reporting the diagnosis to a user.

In some embodiments, the method further includes estimating from the healthy regions of the eye relevant physiological factors including but not limited to: a pupil radius, a pupil eccentricity, a pupil irregularity, a lens fluorescence quantum yield, a lens luminescence intensity ratio, an iris inner radius, an iris outer radius, an iris eccentricity, or an iris border irregularity. In some embodiments, all relevant physiological factors are reported to the user because they are useful measures in, for example, microbial keratitis. In some embodiments, the procedure to determine these relevant physiological factors is altered by the presence of an ulcer.

In some embodiments, a plurality of exposures is collected from each camera and are used to form a composite image (e.g., an averaged image, a 3D image, etc.). In some embodiments, for each of the plurality of exposures, the aperture is at a different position with respect to the eye. In some embodiments, the collecting the first image and the second image is while the device in a first position relative to the eye, and wherein the method further includes collecting a third image of the eye with the first image sensor and a fourth image of the eye while the second image sensor with the device in a second position relative to the eye. In other words, the method includes collecting images of the eye at various angles.

In some embodiments, the method collects the first image and the second image are captured with a spatial resolution of approximately 5 mm⁻¹.

In some embodiments, analyzing the first image and the second image includes calculating a ratio image such that the ratio measured at each pixel is equal to the spectral luminescence intensity ratio normalized to a reference condition.

Fluorescence imaging of reduced nicotinamide adenine dinucleotide (NAD (P) H) and flavin adenine dinucleotide (FAD) have a demonstrated utility in characterizing the metabolic state of tissue via the optical redox ratio, and in discriminating between microbial species. There is significant clinical utility in this measurement, including applications as broad as diagnosis of cancer and infections, but most techniques reported to date require specialized training and equipment making most implementations unsuitable for medical imaging. In this work, a low-cost and robust diagnostic methodology is designed that makes use of the intrinsic fluorescence of NADH and FAD resulting from low-power, near-ultraviolet LED excitation of biological samples. The diagnostic is optimized to distinguish between different microbial species based on spectral data. A detailed performance characterization is provided based on measured B. subtilis fluorescence spectra, and based on a combined NADH-FAD fluorescence model. The performance analysis suggests the measurement is sensitive to changes in the redox ratio (defined here as the concentration ratio of FAD to NADH) over approximately four orders of magnitude. Performance based on B. subtilis data suggests that redox ratios can be measured to within 10% on a per-pixel basis for samples as thin as 10 s of microns at an imaging resolution of approximately 30 mm-1 with exposures on the order of 20 ms. Two representative B. subtilis samples and a green bread mold fungus sample were imaged to demonstrate the proposed technique. Measurements suggest that the luminescence intensity ratio has some dependence on sample thickness, which may be indicative of radiation trapping caused by preferential reabsorption of NADH fluorescence by FAD. Overall, the results suggest that redox imaging of macroscopic tissue and microbe samples is feasible using low-cost optical components and low excitation irradiance which may be suitable for medical imaging for rapid and robust diagnosis of a variety of medical conditions.

In one embodiment, a diagnostic methodology is designed to distinguish between microbial species or metabolic state of biological samples based on the redox ratio measured using near-UV fluorescence spectroscopy of primarily, but not exclusively, NADH and FAD. The chosen method uses ratiometric line-of-sight fluorescence imaging. The collection bands for the diagnostic strategy were chosen based on fluorescence spectra of several microbial species, and performance estimates for the diagnostic were presented. Measurements of B. subtilis fluorescence in two metabolic states, and a wild-type green bread mold fungus were presented and used in the analysis. In some embodiments, a combined NADH-FAD fluorescence model, based on two fluorescent chemicals that play a significant role in mitochondrial activity, is utilized.

An imaging diagnostic for detecting and diagnosing infections of the cornea is detailed herein. Preliminary spectroscopic characterization of microbes was conducted using near ultraviolet excitation and it was found that representative bacterial and fungal species emit with similar intensities (per unit microbe volume) and emission spectra, but are sufficiently distinct that a ratiometric imaging diagnostic can distinguish between sources; this information was used to design a two-band ratiometric fluorescence imaging technique. Additionally, several diagnostic considerations including imaging resolution, UV-radiation exposure, and intrinsic eye fluorescence were assessed and discussed in detail. Finally, diagnostic requirements were formulated and a design was presented and analyzed. Detailed analysis of diagnostic performance was presented to ensure imaging resolution and measurement precision are sufficient to aid in the detection of corneal infections.

Performance analysis suggests the device is capable of making measurements with short exposure durations (on the order of 50 ms) at low irradiance (35 mW/cm²), putting the radiant exposure for a single image orders of magnitude below incidental exposure thresholds for the eye. At these conditions, precise ratio measurements with SNR≥10 on a per-pixel basis at 5 mm⁻¹ spatial resolution are achievable. At this exposure, repeated measurements can be made at a rate of 10 Hz for up to 100 s while remaining at or below the ICNIRP-recommended incidental exposure threshold and not causing damage or injury to the eye.

The imaging device is equally well-suited to the imaging of microbial smears or colonies placed on optical media (e.g., microscope slides) or non-reflecting surfaces on the order of 0.1 mm thick with the same acquisition procedure and equipment described herein. In microbial smears of this nature, the luminescence intensity ratio is closely related to the optical redox ratio, which is used often to characterize cell metabolic function. Smears can be prepared simply by streaking colonies onto a plate, or by mixing with distilled water and allowing to dry. Note that the presence of fluorescent impurities in the water or other solvent used in this procedure may influence the imaging measurement.

The imaging device is also well-suited to the imaging of skin and wounds. Although optically thick, skin cells contain a similar concentration of fluorescent chemicals including NAD(P)H, FAD, melanins, and collagens which are directly excited by the UV source. Since skin is optically thick, measurements are limited to the surface layer and there is little penetration or interference from tissues beneath the surface. The same UV/image exposure, focus procedure, and image acquisition procedure described herein are sufficient for this purpose. Similar to microbe imaging, the optical redox ratio is a measure of cell metabolism that has been used in the study of skin cancers. Similar to imaging of microbial keratitis, infected skin tissue will exhibit a combined fluorescence response including the contribution of the infecting microbes, and changes in the fluorescence intensity and intensity ratio may be used to ascertain the identity of the infection.

In some embodiments, radiometrically characterizing the response of the human eye to ultraviolet excitation and identifying a method for background correction is used to enable unbiased quantitative fluorescence imaging. In some embodiments, intrinsic eye fluorescence is sufficient to focus the device. In other embodiments, a second visible LED is used to focus the device.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

Herzog, J. M., Sick, V. Quantitative Spectroscopic Characterization of Near-UV/visible E. coli (pYAC4), B. subtilis (PY79), and Green Bread Mold Fungus Fluorescence for Diagnostic Applications. J Fluoresc (2023) is incorporated herein by reference in its entirety.

Herzog, Joshua M., and Volker Sick. “Design of a line-of-sight fluorescence-based imaging diagnostic for classification of microbe species.” Measurement Science and Technology 34.9 (2023): 095703 is incorporated herein by reference in its entirety.

Classification of Microbe Species. Fluorescence imaging of certain biochemicals, including flavins and pyridine nucleotides, has utility in characterizing the metabolic state of tissue and in discriminating between microbial species. There is significant clinical utility in this class of imaging techniques but most measurements reported to date require specialized training and equipment rendering most implementations unsuitable for routine medical imaging. Disclosed herein is a low-cost and robust imaging technique using ultraviolet-induced fluorescence of pyridine nucleotides (primarily NADH) and flavins (primarily FAD) in microbial samples. The diagnostic is optimized to distinguish between different microbial species based on previously reported spectral data using a ratiometric imaging approach. A detailed performance analysis is provided that relates the measured fluorescence intensity ratio (FIR) to the relative concentration ratio of NADH to FAD using a simplified spectroscopic model. Analysis suggests the technique is sensitive to changes in the NADH/FAD concentration ratio over several orders of magnitude, with better than 10% FIR precision on a per-pixel basis for microbial smears as thin as 10 s of microns at a resolution of 30 mm-1 and exposures of 20 ms. Representative microbe samples from eight species were imaged to demonstrate the proposed technique. Results show that the FIR varies by an order of magnitude across different species but the intra-species variation is only ˜5% for the conditions used here.

In some embodiments, an additional imaging band is utilized to classify species that contain red pigments or bacteriochlorophyll. Radiative trapping was discussed as a possible limitation of the technique, but no clear evidence for radiative trapping was observed here. Overall, the results suggest that the proposed approach is feasible for rapid, low-cost, and robust characterization of microbial samples.

Disclosed herein is a simple, low-cost ratiometric line-of-sight fluorescence imaging technique to distinguish between microbial species based on spectroscopic investigation. In contrast to confocal and multiphoton microscopy, the line-of-sight technique is a two-dimensional path-integrated imaging measurement; the primary benefits are that it is fast, inexpensive, does not require specialized equipment, and is robust since both fluorescence bands are excited and imaged simultaneously. The shared optical path minimizes bias due to relative motion or differences in source profile and intensity. However, this line-of-sight imaging approach provides only moderate spatial resolution.

The disclosed technique is analyzed using a combined NADH-FAD fluorescence model and demonstrated on 44 microbial smears across 8 species (nonpathogenic strains of B. cereus, B. subtilis, E. coli, M. luteus, P. fluorescens, S. marcescens, and a green bread-mold fungus) to illustrate the utility of the method.

The diagnostic method disclosed herein is designed to target primarily NADH and FAD fluorescence. NADPH, a coenzyme similar to NADH that also plays a role in many metabolic functions, has nearly identical optical properties that make it indistinguishable from NADH. NADPH is likely to be present in samples as well and hence many studies consider NAD (P) H fluorescence, the combination of both NADH and NADPH fluorescence, instead. In this disclosure, the label NADH can be used exclusively for simplicity. However, it is worth noting that measurements may contain some NADPH fluorescence. Since NADPH and NADH have nearly identical fluorescence properties, the combined NADH-FAD model is believed to be representative of NAD (P) H fluorescence. Several additional biochemicals could be observed with this excitation and collection strategy including collagen and melanins; however, NAD (P) H and FAD are believed to be the dominant source of fluorescence in the samples under consideration here. Other common fluorescent biochemicals including proteins like tryptophan and tyrosine do not fluoresce appreciably at the near-UV excitation wavelengths used here.

The line-of-sight fluorescence-based imaging diagnostic disclosed herein is designed to provide a fast, low-cost, and accessible method for assessing microbial smears or other biological samples including tissue samples. In principle, the technique is capable of detecting differences between microbe species and metabolic states due to varying concentrations of fluorescent chemicals, primarily NADH and FAD. Other systematic chemical (e.g., production of pigments or solvation effects) and physical differences (e.g., cell size and shape) between species are expected to further alter absorption and fluorescence properties to aid in classification. The proposed technique is a two-color ratiometric fluorescence imaging measurement, which is intended to be the simplest fluorescence diagnostic that retains some spectroscopy information and is conceptually similar to the optical redox ratio. A detailed overview of the technique and design parameters is presented herein. A methodology for optimizing the collection bands is presented based on fluorescence data from a small subset of samples. Finally, a combined NADH & FAD fluorescence model is presented which will also be used to analyze the diagnostic performance.

Both NADH and FAD can be excited at near-UV wavelengths and have fluorescence quantum yields on the order of several percent in water. NADH and FAD are believed to occur in different concentrations between microbial species in general, and also are known to vary in relative concentration as a function of a cell's metabolic state. Their utility as a marker for metabolic state has been well established. The diagnostic methodology presented here is optimized to detect differences between measured microbe species, but because the FIR is directly related to the optical redox ratio, the technique is suitable for detecting metabolic changes in biological samples as well.

Ratiometric imaging performance. In some embodiments, a simplified performance model is used to optimize diagnostic design. For the ratiometric imaging measurement, the measured FIR at each pixel is simply the ratio of the intensity in the red band to that in the blue band, or

$$R = \frac{{S}_{r}}{{S}_{b}} = \frac{f}{1 - f} \qquad EQN . 33$$

where Sᵢ is the measured signal in band i, and the subscripts b and r represent the blue and red bands, respectively. On the right-hand side, the FIR is rewritten in terms of the fraction f of the total emission that was captured in band r, and it is assumed that the collection bands are complimentary to ensure the maximum signal is collected. For simplicity, the collection bands are assumed to be square, so f is determined only by the cutoff wavelength of the beamsplitter.

The FIR precision is estimated via first-order uncertainty propagation as

$${( \frac{{s}_{R}}{R} )}^{2} = {( \frac{{s}_{{S}_{b}}}{{S}_{b}} )}^{2} + {( \frac{{s}_{{S}_{r}}}{{S}_{r}} )}^{2} \qquad EQN . 34$$

where sₓ is the precision index (single-shot standard deviation) in the variable x. For a typical image sensor,

$${s}_{Si}^{2} = {N}_{r , i}^{2} + {S}_{i} \qquad EQN . 35$$

where N_{r,i} is the read noise. In the shot-noise limit, N_{r,i}«Sᵢ and s_{S}_{ᵢ}==\sqrt{Sᵢ} such that

$${( \frac{{s}_{R}}{R} )}^{2} \approx \frac{1}{{S}_{b}} + \frac{1}{{S}_{r}} \qquad EQN . 36$$

For a total emitted signal S=Sᵣ+S_{b} with signal-to-noise ratio (SNR) of SNR₀, the FIR precision can be rewritten as

$$\frac{{s}_{R}}{R} = \frac{1}{\sqrt{{SNR}_{0}}} \frac{1}{\sqrt{f - {f}^{2}}} = \sqrt{\frac{1 + R}{f {SNR}_{0}}} \qquad EQN . 37$$

The FIR precision is clearly optimized when f=0.5, or rather when the signal is split equally between both sensors.

The precision with which relative species concentration can be determined depends on the sensitivity of the FIR to concentration. For a continuous function, e.g., the concentration ratio χ(χ=xFAD/xNADH where x is mole fraction), the measurement precision is given by

$$\frac{{s}_{χ}}{χ} = \frac{1}{{β}_{χ}} \frac{{s}_{R}}{R} \qquad EQN . 38$$

where βχ is the dimensionless sensitivity of the FIR to χ:

$${β}_{χ} = \frac{d log R}{d log χ} = \frac{x}{R} \frac{dR}{d χ} \qquad EQN . 39$$

For the discrete case where the FIR is used to identify an organism, the FIR precision is instead compared to the typical variation in FIR between a representative group of species to estimate an error metric as

$$E = \frac{{s}_{R}}{{Δ}_{R}} ∝ \frac{\sqrt{f}}{{{Δ}_{R} ( 1 - f )}^{3 / 2}} \qquad EQN . 40$$

where Δ_{R} is a measure of the variation in FIR between a representative group of samples (e.g., the standard deviation).

The optimum cutoff wavelength is chosen as the value that minimizes the relative error quantity E calculated from measured fluorescence spectra of E. coli, B. subtilis, and a wildtype green bread mold fungus. Specifically, for each cutoff wavelength, R and s_{R} are calculated for each spectrum. The standard deviation of R is calculated to estimate Δ_{R}, and the mean s_{R} is calculated for comparison with Δ_{R}. This procedure is repeated for a range of potential cutoff wavelengths, and the cutoff wavelength with the smallest E value is chosen. This procedure effectively calculates the mean FIR precision for each cutoff wavelength and compares it to the variation in FIR across species, and so directly identifies a cutoff wavelength suitable for ratiometric imaging to distinguish between samples.

In some embodiments, a second optimization is performed using the model NADH-FAD spectrum. However, the NADH and FAD models are derived from characterization data of the chemicals in aqueous solution and do not consider changes in the chemical environment or cell morphology between species. In particular, changes in the chemical environment can cause small shifts in absorption and fluorescence feature locations which may be useful in distinguishing bacteria. As such, the measured fluorescence spectra are believed to be a better indicator of changes in microbial fluorescence and are preferred for diagnostic design. The model spectrum instead provides a convenient, species-agnostic tool for performance analysis where the impact of additional parameters including excitation wavelength can be assessed.

Combined NADH-FAD fluorescence model. A quantitative, combined NADH and FAD fluorescence model is used to analyze the performance of the proposed diagnostic technique. The model is based on aqueous solutions of NADH and FAD, but includes small changes in fit parameters to better match observed E. coli spectra which are believed to be representative of NADH and FAD in biological samples. This model provides a convenient method to calculate absorption and fluorescence spectra. The primary limitations of the model are that differences in the chemical environment between microbe samples are neglected, and that cell morphology is ignored. The chemical environment can cause small shifts in absorption and fluorescence features and changes in collisional quenching properties, while cell morphology and inhomogeneity impact radiative transfer and trapping; both effects are expected to be species dependent. However, these simplifications are necessary to provide a useful model for analysis, and are believed to be appropriate for general performance quantification of nonpigmented samples such as E. coli and B. subtilis.

Mathematically, each molecule is represented by a single one-dimensional configuration coordinate model implemented as a harmonic oscillator. The NADH and FAD absorption and fluorescence spectra are thus each described by a single electronic transition, and absorption and fluorescence spectra are calculated in the Condon approximation. Since FAD has two distinct absorption peaks, likely arising from two electronic transitions, only the longer wavelength peak (near 450 nm) was included in the fit. For shorter wavelength near-UV absorption, experimental data from is interpolated instead and the fluorescence quantum yield (FQY) is assumed to be constant. The model parameters are listed in Table 5.

TABLE 5
Best-fit values for NADH and FAD using 1D-CCM. The FQY (Φ)
and radiative deactivation rate (A) for each molecule is include.

| | NADH | FAD | units |
|---|---|---|---|
| T″ | 25,220 | 20,060 | cm⁻¹ |
| v′ | 756 | 775 | cm⁻¹ |
| v″ | 818 | 843 | cm⁻¹ |
| δ_{q} | 2.8 | 2.32 | — |
| W | 500 | 500 | cm⁻¹ |
| μ′ | 3.94 | 4.32 | Debye |
| μ″ | 3.63 | 2.95 | Debye |
| Φ | 0.02 | 0.04 | — |
| A | 48.5 | 17.6 | MHz |

In the Condon approximation, the absorption cross-section and fluorescence rate are given by

$$σ ( ω ) = \frac{4 {π}^{2} αω}{3 {e}^{2}} {\vert {μ}^{′} \vert}^{2} {\sum }_{ij} \frac{{e}^{- \frac{{G}_{i}^{′}}{{k}_{B} T}}}{{Q}_{v}^{′} ( T )} {\vert 〈 i \vert j 〉 \vert}^{2} ℒ ( ω - {ω}_{ij} ; W ) \qquad EQN . 41$$

$$and$$

$$k ( ω ) = \frac{4 {ω}^{3} α}{3 {c}^{2} {e}^{2}} {\vert {μ}^{″} \vert}^{2} {\sum }_{ij} \frac{{e}^{- \frac{{G}_{j}^{″}}{{k}_{B} T}}}{{Q}_{v}^{″} ( T )} {\vert 〈 i \vert j 〉 \vert}^{2} ℒ ( ω - {ω}_{ij} ; W ) \qquad EQN . 42$$

respectively, where ω is the photon angular frequency, α is the fine-structure constant, c is the speed of light, e is the electron charge, μ is the transition dipole moment, Gₗ is the vibrational energy, k_{B} is the Boltzmann constant, Qᵥ is the vibrational partition function, and L is the lineshape function. Here, the lineshape is assumed to be Gaussian with standard deviation W. The transition frequency between levels i and j in the model is given by

$${ω}_{ij} = {T}^{″} + {G}_{j}^{″} - {G}_{i}^{′} \qquad EQN . 43$$

where Tis the electronic energy; the ground state electronic energy is defined to be zero for simplicity. The superscript prime (′) and double prime (″) are used to indicate the ground and excited states, and [symbol] i|j [symbol] represents the Franck-Condon factor; a harmonic oscillator model is used to simplify the calculation of [symbol] i|j [symbol] .

Imaging Demonstration. A ratiometric imaging demonstration was performed for multiple smears from eight different plated microbial cultures. Smears were prepared over the course of two weeks and sampled from different regions to ensure the measurements represent a range of metabolic states. The microbial smears were prepared by streaking approximately 1-3 mm³ of the culture onto a black anodized aluminum substrate and spreading it over a small area with an inoculation loop. The samples were additionally mixed with approximately 20 μL of distilled water and dried to create a more uniform thickness across the sample. Imaging exposure was varied between 200 and 500 ms to ensure similar imaging precision was achieved with each smear. A series of 10 images was taken and averaged for each sample, and a background correction was performed with the LED turned off using an average over 10 images. A measurement of the LED irradiance profile was made using fluorescence of white paper, and raw signal images were normalized by the LED profile to account for variation in irradiance and collection efficiency (e.g., vignetting). Finally, a mask was generated using an automated threshold technique; regions where the LED irradiance was below 50% of the center value were also masked. Measurements of the cleaned substrate were recorded and showed negligible luminescence intensity.

Fluorescence spectra. Fluorescence spectra of several microbial samples were measured using a 365-nm LED module (ThorLabs M365LP1) and UV-cutoff filter (ThorLabs FELH0400). Smears were prepared as described herein. The inoculated substrate was placed approximately 300 mm in front of the focused LED, and was exposed to UV-radiation at approximately 30 mW/cm². Fluorescence was collected using a fiber-coupled lens (ThorLabs FMA810-635) placed behind the sample and substrate, and directed into a pocket spectrometer (Ocean Optics USB2000+). The spectrometer was integrated for 20 seconds and averaged over 30 samples. Measurements were background subtracted and corrected for relative spectral response using a quartz-tungsten halogen lamp (World Precision Instruments, D2H). Spectra were smoothed using a first-order Savitzky-Golay filter with approximately 10.5 nm window to reduce noise.

Example Results. The optimization procedure disclosed herein was performed to identify the optimum cutoff wavelength based on a limited set of microbe fluorescence spectra. The optimized collection bands were used to design and assemble an implementation of the diagnostic, and the system was used to image several microbe smears to illustrate the concept. Manufacturer-provided data was used to estimate performance for the diagnostic and performance estimates were made based on the combined NADH-FAD fluorescence model.

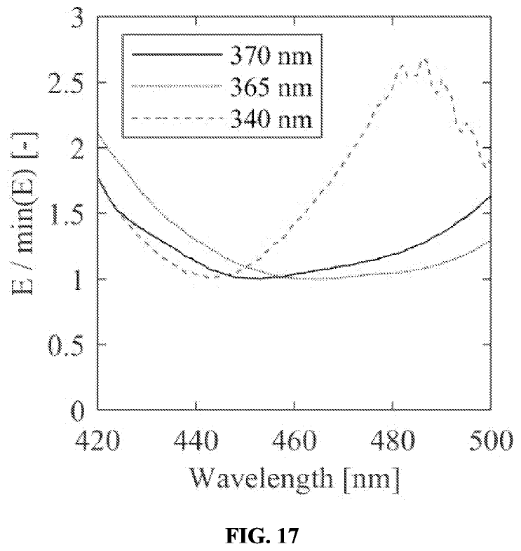

Optimized collection strategy. With reference to FIG. 17, the calculated error metric is plotted as a function of cutoff wavelength using 340 and 370 nm LEDs (with approximately 15-20 nm bandwidth) for excitation of B. subtilis, E. coli, and green bread mold fungus. A third error metric was calculated from spectra acquired here with 365 nm excitation for the seven bacteria species described herein. The calculation considered only emission from 400 to 530 nm to, among other things, to avoid the influence of additional red pigments or fluorophores in the current samples. The error metric was normalized by its minimum value at each excitation wavelength since it does not include absolute fluorescence intensity. From the plot, increasing excitation wavelength from 340 nm increases the optimum cutoff wavelength from 440 to 450-460 nm, consistent with the increase in green fluorescence. Here, the 365 nm LED was chosen for its combination of increased output power and increased green fluorescence compared to the 340 nm LED module, and the optimized cutoff wavelength is approximately 460 nm.

Band-pass filters were not selected for this application for two reasons. First, stock long- and short-pass filters offer more flexibility in terms of cutoff wavelength selection and can provide transmission bands that are approximately flat with high peak transmission. Stock components are used here exclusively because low-cost and high-availability of optical components is a primary benefit of fluorescence imaging compared to other technologies. Second, the optimal upper cutoff wavelength of the green fluorescence band is not immediately clear from the data presented here; this point will be discussed further herein.

Diagnostic Implementation Example. An example implementation of the optimized imaging strategy was assembled from the following components. Two identical, 1.6 MP CMOS cameras (ThorLabs CS165MU1) are used to image fluorescence with a 458 nm dichroic beamsplitter (Semrock FF458-Di02). A 365 nm LED with peak power of 2 W is used for excitation (ThorLabs M365LP1), and a 389 nm dichroic beamsplitter (Semrock FF389-Di02) is used to direct the UV emission towards the object plane. The LED emission is restricted with a bandpass filter (Semrock Hg01-365-25). The two image sensors share an achromatic lens (Edmund Optics #65-976) to ensure magnification is consistent between image bands. The LED is focused slightly onto the object plane using a condenser lens (Edmund Optics #48-304). Reflected UV light is rejected using a UV-rejection filter placed in front of the imaging lens (ThorLabs FELH0400). A short-pass filter (ThorLabs FESH0450) and long-pass filter (ThorLabs FELH0450) were used to restrict the collection bands of the blue and red cameras, respectively. The condenser lens and imaging lens were analyzed and selected based on the results of a raytracing calculation.

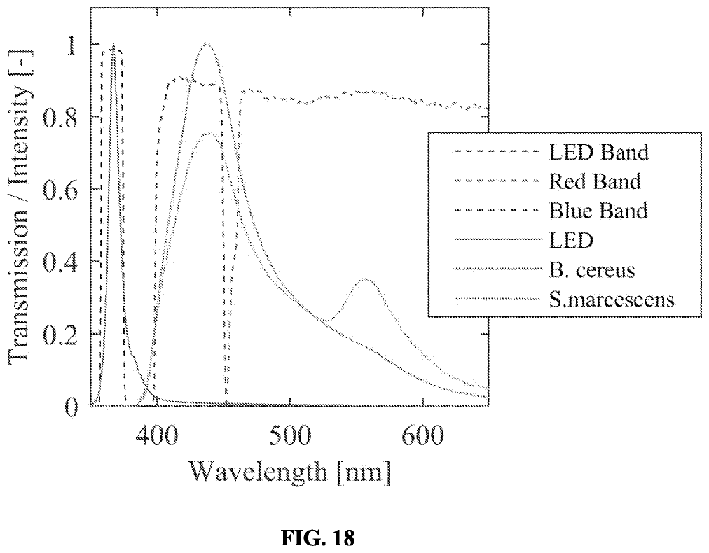

The transmission bands, calculated from manufacturer-provided data, are shown in FIG. 18 with the LED emission spectrum and two fluorescence spectra (B. cereus and S. marcescens) superimposed for reference. The transmission bands for the image sensors include both beamsplitters, the UV-rejection filter, and the long- or short-pass filter as appropriate. The LED band includes the UV beamsplitter and band-pass filter. The camera quantum efficiency is excluded from the plot and is between 50 and 70% in the 400 to 600 nm wavelength range. Each lens is anti-reflection coated, so losses are typically <0.5% per surface and are excluded from the remaining analysis. Comparing the transmission bands to the fluorescence spectra, the collection bands correlate well with relative changes in fluorescence spectra between species

An f/2.0, 50 mm focal length lens was chosen for the imaging lens and the magnification is chosen to be approximately −0.2. This results in an object plane distance of 300 mm and an approximately 25 mm field of view which is suitable for imaging of samples on, e.g., microscope slides. The object plane pixel size is approximately 17 μm, corresponding to a best-case spatial resolution of approximately 30 mm⁻¹.

Performance Predictions. Performance factors including collection fraction, the NADH and FAD collection efficiencies per band, the B. subtilis fluorescence collection efficiency per band, the excitation irradiance, and the collection volume were calculated and are listed in Table 6. A representative fluorescence coefficient for bacterial smears is included. These parameters are sufficient to estimate performance for imaging of pure NADH-FAD mixtures and for typical bacterial smears assuming the fluorescence spectra are similar to that of B. subtilis. The radiometric parameters are used to estimate imaging precision for the model NADH-FAD mixture, and for a representative bacterial smear.

TABLE 6
Performance parameters for implantation according to one embodiment.
The collection fractions ηₓ include optical efficiency,
filter band transmission, and sensor quantum efficiency.

| Symbol | Description | Value | Units |
|---|---|---|---|
| k_{f} | Fluorescence coeff. | 0.05 | mm⁻¹ |
| η_{NADH, r} | NADH red fraction | 0.23 | — |
| η_{NADH, b} | NADH blue fraction | 0.26 | — |
| η_{FAD, r} | FAD red fraction | 0.58 | — |
| η_{FAD, b} | FAD blue fraction | 2 · 10⁻⁵ | — |
| η_{BSub, r} | B. subtilis red fraction | 0.39 | — |
| η_{BSub, b} | B. subtilis blue fraction | 0.14 | — |
| \|M\| | Magnification | 0.2 | — |
| f_{#} | Lens f-number | 2.0 | — |
| Ω/4π | Collection fraction | 4.3 · 10⁻⁴ | — |
| lₒ | Pixel size | 17.25 | μm |
| I″ | LED irradiance | 30 | mW/cm² |
| Nᵣ | Camera read noise | 4.0 | e⁻ |
| C_{AD} | Analog-to-digital gain | 10.74 | e⁻/ADU |

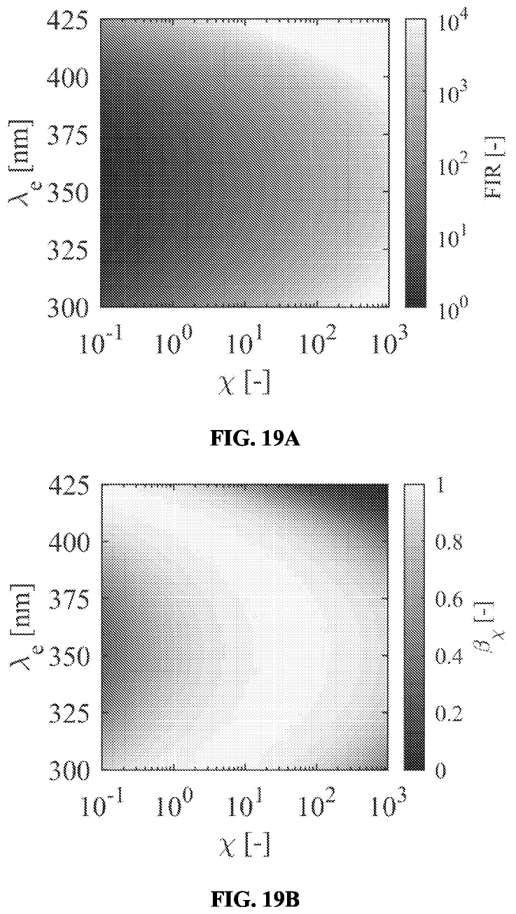

NADH-FAD ratio and sensitivity. The combined NADH-FAD fluorescence model was used to calculate the ratio calibration function R and sensitivity βχ as a function of excitation wavelength and FAD/NADH concentration ratio χ. The ratio and sensitivity are plotted in FIGS. 19A and 19B. From the plots, the minimum ratio is approximately 0.8, which is the ratio of pure NADH. As the FAD fraction is increased, R∝χ since the vast majority of the FAD emission is captured within the red band. At sufficiently high values of χ, R again approaches a constant value equal to the ratio of pure FAD. The sensitivity over the majority of the plotted range for χ>10⁻¹ is βχ≈1, which is expected for R∝χ.

At the design wavelength of 370 nm, the measurement is sensitive to χ when χ≥10⁻¹. FIRs on the order of 10² can be measured with typical image sensors, which suggests that it is feasible to perform this measurement for 10⁻¹≤χ≤10² at λe≈370 nm. Smaller FAD ratios can be measured by tweaking the excitation wavelength to preferentially excite FAD more efficiently, e.g., by increasing λₑ; the dynamic range could also be altered by using different integration durations for each fluorescence band. Notably, the technique is sensitive over approximately four orders of magnitude of χ.

NADH-FAD measurement limits. Since the imaging diagnostic provides a line-of-sight measurement, the optical path length or thickness of the sample has a strong impact on imaging performance. Performance also depends strongly on the total concentration of NADH and FAD in the sample. In addition to the concentration ratio χ, performance depends only on the product htn where h is the thickness of the sample, t is the exposure duration, and n is the total number density of NADH and FAD, assuming the sample is thin.

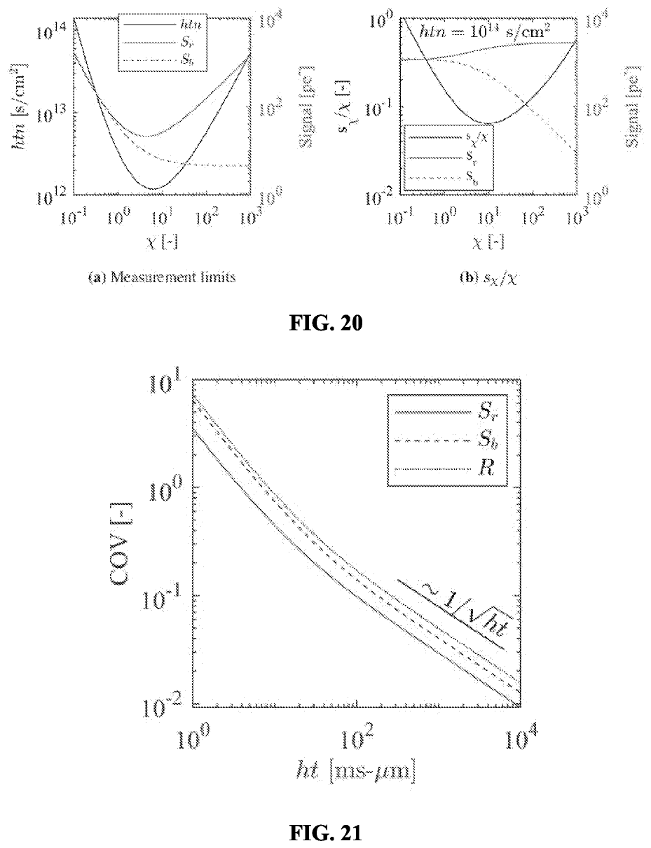

The measurement limit is calculated here as the value of htn as a function of χ such that the signal-to-noise ratio of the χ measurement is equal to unity, and the result is plotted in FIG. 20A. From the plot, precision is optimized for χ≈6, with htn approximately 10¹² s/cm². For a 1 second exposure duration and a 0.1 mm thick sample, this corresponds to a limit of n≈10¹¹ mm⁻³ or a concentration of 0.2 μM. The measured signal in electrons is also plotted in FIG. 20A; the cameras have a full-well capacity of approximately 11,000 e⁻.

The calculated coefficient of variation (COV) in the measured χ value is plotted in FIG. 20B for a fixed concentration of htn=10¹⁴ s/cm². For an exposure duration of 1 second and sample thickness of 0.1 mm, this corresponds to n≈1013 mm⁻³ or a ˜20 μM solution of NADH and FAD. At these conditions, it is estimated that χ can be measured to within 10% per pixel over approximately two orders of magnitude. Comparing the signal intensities per channel, the red band actually remains relatively constant and changes by only a factor of approximately 2.5 for any value of χ. The red band intensity is thus a good indicator of total concentration and is used along with the ratio measurement, in some embodiments, to provide an absolute concentration measurement for both NADH and FAD.

Signal and ratio precision for microbe film. Performance for the bacterial smear is a function only of the effective film thickness h and integration duration t for a densely packed microbial layer since n is approximately fixed by cell geometry. The estimated ratio and fluorescence intensity COVs are plotted as a function of ht in FIG. 21 for B. subtilis assuming a fixed fluorescence coefficient of k_{f}=0.05 mm⁻¹. From Table 6, the B. subtilis FIR is fixed at 1.85, which corresponds to χ≈0.27 from the combined NADH-FAD model.

With continued reference to FIG. 21, near ht≈30 ms-μm, the measurement becomes shot-noise limited and performance becomes proportional to the square-root of signal intensity; the ratio COV is approximately 0.4 when this occurs. Typical COVs for the FIR for ht≥10² ms-μm are less than 0.2 which suggests that good imaging precision is feasible even for relatively low exposures and thin microbe layers. For example, at a typical thickness of 0.1 mm and exposure duration of 10 ms, s_{R}/R≈0.05 with approximately 1000 e⁻ captured in the red band which is well below the saturation threshold. Although the selected cameras are incapable of hardware binning, software binning or averaging could be used to improve measurement precision as needed to determine an aggregate or lower-resolution FIR. An approximate minimum sample thickness for which reasonable quality imaging results can be obtained is, in some embodiments, 10 μm for a 20 ms exposure, resulting in an FIR COV of approximately 10%.

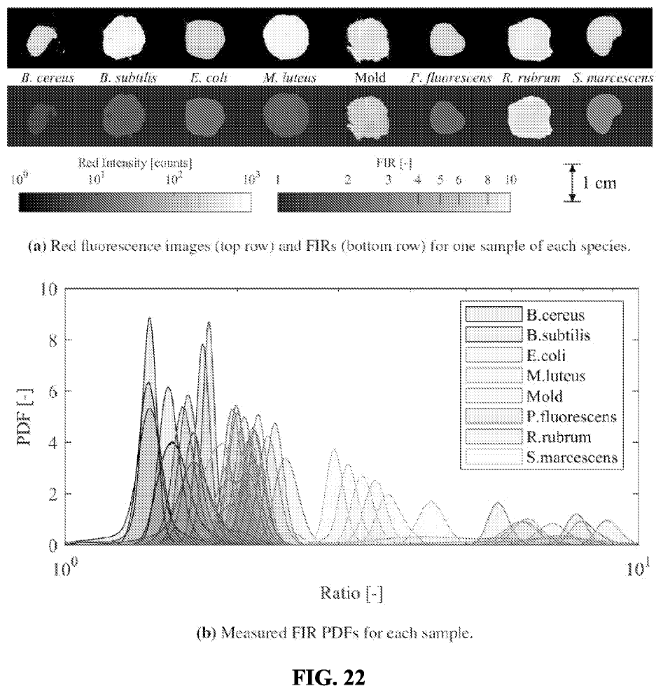

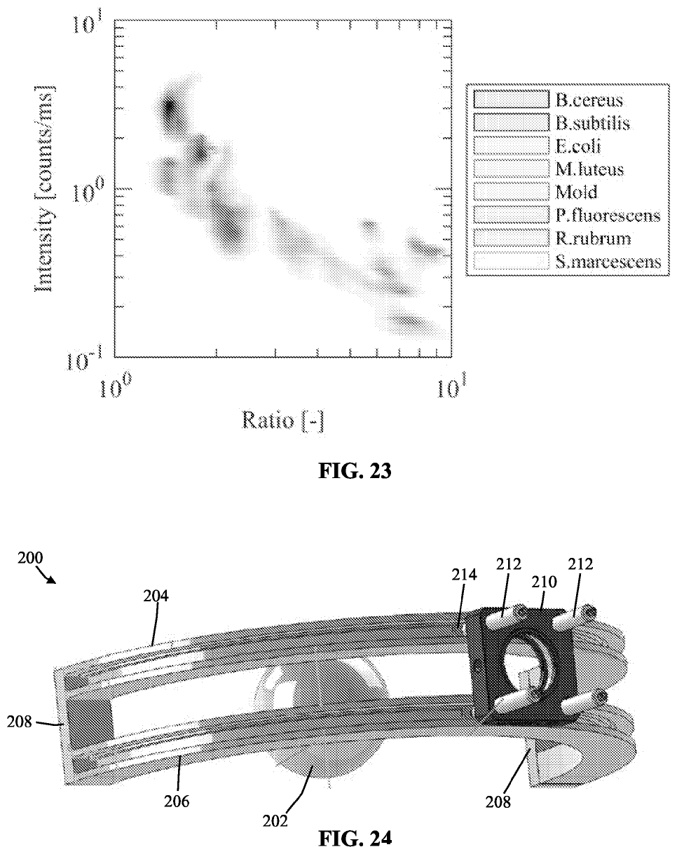

Imaging demonstration. An imaging demonstration was performed on 4-6 samples from each microbial culture over the course of two weeks. The average blue band fluorescence images and FIR images for a representative sample from each culture are shown in FIG. 22A, with the measured ratio probability distribution functions (PDFs) plotted in FIG. 22B.

Comparing the FIR PDFs, there is significant variation across species with values ranging from approximately 1.5 (B. cereus) to approximately 9 (R. rubrum). The coefficient of variation (COV) between samples of the same species is on the order of 5% for most species (B. cereus, B. subtilis, E. coli, M. luteus, and P. fluorescens), while the remaining species have COVs closer to 15%. Species with higher FIR COVs (R. rubrum, S. marcescens, and green bread mold) tend to have larger FIR values. This is not surprising as the higher FIR values correspond to weaker blue-band fluorescence intensity, which makes the FIR sensitive to small changes in blue band fluorescence intensity. It is also possible that the intra-species FIR variation could be influenced by background or stray light where the fluorescence intensity is weak, although there is no clear evidence of this in the data. Within a given sample, however, the per-pixel variation in FIR is on the order of 10% based on the full-width at half maximum (FWHM) of the distribution functions (corresponding to ˜4% standard deviation assuming a normal distribution). The FIR variation within an image is significantly larger than that expected from thermal and shot noise, suggesting that the FIR distributions are dominated by inhomogeneity in the sample.

R. rubrum and S. marcescens exhibit significantly higher FIR values than the other bacteria samples which is likely influenced by the presence of additional pigments. For example, S. marcescens contains the red pigment prodigiosin, the production of which is known to depend on growth phase and available nutrients. Prodigiosin is known to strongly absorb blue and green light and fluoresce at red wavelengths, resulting in an increase in the measured FIR. Likewise, R. rubrum produces bacteriochlorophylls and carotenoid when photosynthesis-active and is known to absorb strongly at UV, visible (˜590 nm) and infrared wavelengths. Bacteriochlorophyll is also fluorescent and emits between ˜750 and 850 nm with a quantum yield on the order of 0.1, which could account for the large FIR and red band emission intensity.

A detailed design and analysis of an imaging strategy is disclosed herein to distinguish between microbial species based on NADH and FAD fluorescence. Performance analysis indicates that even very thin layers can be imaged with good precision, and the technique was demonstrated and data was presented for 44 microbial smears. Since this method uses line-of-sight imaging and excitation, it is possible that radiative trapping could impact the result. Radiative trapping in most bacteria samples here is expected to lead to reduction in blue-band intensity through reabsorption by FAD, increasing the FIR. This would be evident in the data as a dependence of FIR on blue-band intensity within a single sample which is not clearly evident in the data presented here. Joint PDFs of blue-band intensity rate (signal per unit exposure time) and FIR are plotted in FIG. 22. From the plot, there is an apparent relationship between FIR and blue-band intensity for several samples of S. marcescens, R. rubrum, and green bread mold fungus; however, because the blue band intensity is so weak for these samples, it is also possible that the observed dependence is a result of a small uncorrected background luminescence from the substrate. Indeed, this possible dependence is apparent only in the samples with the lowest fluorescence intensity. On average across all samples tested, the measured FIR is slightly negatively correlated with blue-band intensity within an image ( [symbol] p [symbol] ≈−0.2).

The imaging implementation analyzed herein used two imaging bands with a cutoff near 460 nm. However, fluorescence spectra and imaging results suggest there is a significant red fluorescence component in the pigmented species. The red fluorescence component likely contributes to the increased separation in FIR between the pigmented and nonpigmented bacteria. In some embodiments, the addition of a third imaging band for the red fluorescence component improves classification accuracy, particularly to distinguish between pigmented and unpigmented bacteria.

An imaging technique disclosed herein was designed to distinguish between microbial species based on the relative concentrations of NADH and FAD using near-UV fluorescence. The method uses ratiometric line-of-sight fluorescence imaging. The collection bands for the diagnostic strategy were chosen based on fluorescence spectra of several microbial species. Measurements of 44 microbial smears across 8 species were presented to demonstrate the technique. A performance model based on NADH and FAD fluorescence was presented and performance estimates were made. Performance analysis indicates that microbial smears can be imaged with good precision at short exposure durations on the order of 10 ms, and that the FIR measured by the proposed diagnostic is sensitive to the relative FAD fraction over a several orders of magnitude based on the model. Imaging results show that FIR varies predominantly with species, suggesting that the method may be suitable for classifying microbial samples. In some embodiments, a third imaging band is included to classify species containing red pigments. Radiation trapping was discussed as a potential issue with the measurement, but no clear evidence of this was observed in the imaging data.

Computed Tomography Approach. In some embodiments, a plurality of images are captured at various positions relative to the target (e.g., eye) to form a 3D composite image. In some embodiments, the eye is imaged at five or more different incidence angles (e.g., −90, −45, 0, 45, and 80 degrees relative to the optical axis). Then, for example, a series of 10 images are captured at each orientation. In other words, image sensors capture a plurality of images to form a composite image (e.g., a 3D image, a CT image).

With reference to FIG. 24, a positioning assembly 200 is illustrated for positioning an imaging device (e.g., device 10, device 100) with respect to an eye 202. The positioning assembly 200 includes a first rail 204, a second rail 206, and connector plates 208 connecting ends of the rails 204, 206 together. A camera bracket 210 is slidably secured to the rails 204, 206. In the illustrated embodiment, the camera bracket 210 is slidably secured to the rails 204, 206 with rods 212 and washers 214. The camera bracket 210 is movable with respect to the rails 204, 206 and the eye 202. In some embodiments, the camera bracket 210 is movable approximately 70 degrees inwards and approximately 110 degrees outwards. In some embodiments, the positioning assembly 200 is utilized to position the imaging device (e.g., device 10, device 100) in various positions relative to an eye in order to capture a series of images to create a 3D composite image.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

1. A device comprising:
a frame including an aperture defining an aperture axis;
an imaging lens aligned with the aperture axis;
a first image sensor with a first imaging axis aligned with the aperture axis;
a second image sensor with a second imaging axis;
a light source configured to emit a light along a source axis;
a first beam splitter positioned at an intersection of the aperture axis and the source axis; and
a second beam splitter positioned at an intersection of the first imaging axis and the second imaging axis.

2. The device of claim 1, further including
a first filter aligned with the first image sensor;
a second filter aligned with the second image sensor; and
a third filter aligned with the source axis.

3. The device of claim 2, further including a collimating lens aligned with the source axis.

4. The device of claim 1, further including a cutoff filter positioned between the imaging lens and the first beam splitter.

5. The device of claim 1, wherein the imaging lens is an achromatic lens, a spherical lens, or an aspherical lens.

6. The device of claim 1, wherein the imaging lens is positioned between the first beam splitter and the second beam splitter along the aperture axis.

7. The device of claim 1, wherein the imaging lens has a focal length of 50 mm.

8. The device of claim 1, wherein the imaging lens is transmissive within a range of 400 nm to 700 nm.

9. The device of claim 2, wherein the first filter is a 450 nm long-pass filter; and wherein the second filter is a 450 nm short-pass filter.

10. (canceled)

11. The device of claim 2, wherein the first filter is positioned within a threaded aperture of the first image sensor, and the second filter is positioned with a threaded aperture of the second image sensor.

12. The device of claim 1, wherein the second beam splitter has a cutoff wavelength of 458 nm.

13. The device of claim 1, wherein the light source is an ultraviolet light emitting diode.

14. The device of claim 1, wherein the source axis is orthogonal to the aperture axis.

15. The device of claim 1, wherein the source axis is parallel to the second imaging axis.

16. The device of claim 1, wherein the frame includes a first cage, a second cage, a plurality of cage rods extending from the first cage and the second cage.

17. The device of claim 16, wherein the first beam splitter is positioned within the first cage and the second beam splitter is positioned within the second cage;
wherein the first image sensor and the second image sensor are coupled to the second cage;
wherein the light source is coupled to the first cage; and
wherein the imaging lens is positioned between the first cage and the second cage.

18. (canceled)

19. (canceled)

20. (canceled)

21. The device of claim 1, wherein the second imaging axis is orthogonal to the first imaging axis.

22. The device of claim 1, wherein a distance between the imaging lens and the first image sensor is 60 mm.

23. The device of claim 1, wherein the device is a hand-held line-of-sight diagnostic tool.

24. The device of claim 1, further including a third image sensor with a third imaging axis, and a third beam splitter positioned at an intersection of the first imaging axis and the third imaging axis.

25.-53. (canceled)
